(19) United States
(12) Patent Application Publication
(10) Pub. No.: US 2024/0374517 A1
(43) Pub. Date: Nov. 14, 2024

(54) LIPIODOL FORMULATION FOR TRANSARTERIAL CHEMOIMMUNOEMBOLIZATION

(71) Applicant: Northwestern University, Evanston, IL (US)

(72) Inventors: Heegon Kim, Evanston, IL (US); Dong Hyun Kim, Evanston, IL (US)

(21) Appl. No.: 18/691,863

(22) PCT Filed: Sep. 19, 2022
(86) PCT No.: PCT/US22/76676

§ 371 Date: Mar. 13, 2024

Related U.S. Application Data
(60) Provisional application No. 63/261,320, filed on Sep. 17, 2021.

(51) Int. Cl. A61K 9/107; A61K 31/704; A61K 33/18; A61K 45/06; A61K 47/10; A61K 47/44; A61P 35/00

(52) U.S. Cl. CPC A61K 9/107; A61K 31/704; A61K 33/18; A61K 45/06; A61K 47/10; A61K 47/44; A61P 35/00

(57) ABSTRACT

Disclosed are compositions for use in locoregional delivery, including intra-tumoral and transarterial chemoembolization (TACE). Also disclosed, are methods for treating a subject in need thereof with the compositions described.

### CROSS-REFERENCE TO RELATED APPLICATIONS

This application claims priority to, and the benefit of, U.S. Application No. 63/261,320 filed Sep. 17, 2021, the content of which is incorporated herein by reference in its entirety.

### STATEMENT REGARDING FEDERALLY SPONSORED RESEARCH

Not applicable.

### BACKGROUND

Immunotherapy has tremendous potential to improve cancer treatment. Thus far, there have been few attempts to deliver immunotherapy via transarterial administration. Whereas lipiodol emulsion is commonly used for transarterial administration of drugs, it is unstable and causes systemic leakage of drugs. Thus, incorporation of immunotherapy agents in the conventional emulsion may increase the risk for systemic immune-related adverse events. Therefore, there is significant need for development of a method to safely incorporate immunotherapy for transarterial delivery. Drug eluting beads (DEB) are used for delivery of doxorubicin and can lower its systemic exposure compared to lipiodol emulsion; however, DEBs have limited efficacy in delivering other molecules such as immunotherapy agents.

Lipiodol exhibits transient and plastic embolic effects and it facilitates localized delivery of anticancer drugs into hepatocellular carcinoma (HCC). However, substantial instability and heterogeneity of the lipiodol-based emulsions and potential risk for systemic adverse events due to systemic leakage pose major setbacks in using them for transarterial chemoembolization (TACE). That said, significant safety considerations are required when additionally incorporating immuno-boosting agents to avoid causing adverse events upon systemic exposure using the TACE procedure.

Therefore, there is a need for the development of a formulation with improved stability and that can minimize systemic leakage. This will not only improve the current standard of care using chemotherapy drugs but also allow safe incorporation of potent immunotherapy agents into the procedure.

### SUMMARY OF THE INVENTION

In some embodiments of the current disclosure, methods of locoregionally delivering one or more bioaffecting agents to a subject in need of treatment for cancer are provided. In some embodiments, the methods comprise: i) preparing an emulsion composition comprising lipiodol, a copolymer surfactant, and one or more bioaffecting agents; ii) administering the emulsion composition locoregionally to a subject at a region of the cancer thereby delivering an effective amount of the bioaffecting agents. In some embodiments, the emulsion composition is delivered by transarterial administration. In some embodiments, the emulsion composition is delivered by intratumoral administration. In some embodiments, the one or more bioaffecting agents comprises a chemotherapeutic drug. In some embodiments, the chemotherapeutic drug is doxorubicin. In some embodiments, the one or more bioaffecting agents comprises an immunotherapeutic or immune boosting agent. In some embodiments, the immunotherapeutic is a toll-like receptor (TLR) agonist. In some embodiments, the emulsion composition comprises at least two bioaffecting agents, wherein the first bioaffecting agent is a cancer therapeutic and the second bioaffecting agent is an immune boosting or immunotherapeutic agent. In some embodiments, the one or more bioaffecting agents comprise doxorubicin and a toll-like receptor (TLR) agonist.

In some embodiments, the emulsion composition comprises the copolymer surfactant at a concentration of about 1-30% (weight/volume), preferably about 20% weight/volume. In some embodiments, the emulsion composition has at least 500-fold increased stability as compared to a composition without the copolymer surfactant. In some embodiments, the one or more bioaffecting agents remains localized to the tumor for a suitable amount of time to treat the cancer.

In some embodiments of the current disclosure, methods of treating cancer are provided. In some embodiments, the methods comprise: (i) administering an emulsion composition comprising lipiodol, a copolymer surfactant, and one or more bioaffecting agents in an amount effective to treat the cancer. In some embodiments, the emulsion composition is delivered by transarterial administration. In some embodiments, the emulsion composition is delivered by intratumoral administration. In some embodiments, the emulsion composition comprises the copolymer surfactant at a concentration of about 1-30% (weight/volume), preferably about 20% weight/volume. In some embodiments, the one or more bioaffecting agents comprises a chemotherapeutic drug. In some embodiments, the chemotherapeutic drug is doxorubicin. In some embodiments, the one or more bioaffecting agents comprises an immunotherapeutic or immune boosting agent. In some embodiments, the immunotherapeutic is a toll-like receptor (TLR) agonist. In some embodiments, the emulsion composition comprises at least two bioaffecting agents, wherein the first bioaffecting agent is a cancer therapeutic and the second bioaffecting agent is an immune boosting or immunotherapeutic agent. In some embodiments, the one or more bioaffecting agents comprise doxorubicin and a toll-like receptor (TLR) agonist. In some embodiments, the emulsion composition is made by the method comprising: i) mixing the hydrophilic surfactant and one or more bioaffecting agents: ii) emulsifying the mixture of step i) in lipiodol for a sufficient time to produce an emulsion composition.

In some embodiments of the current disclosure, methods of activating an innate and/or adaptive immune response to a tumor in a subject in need thereof are provided. In some embodiments, the methods comprise: (i) administering an emulsion composition comprising lipiodol, a hydrophilic surfactant and one or more bioaffecting agents, wherein the one or more bioaffecting agents comprises at least one immune boosting or immunotherapeutic agents; and wherein the composition activates an innate and/or adaptive immune response to the tumor as compared with conventional composition. In some embodiments, the emulsion composition is delivered by transarterial administration. In some embodiments, the emulsion composition is delivered by intratumoral administration. In some embodiments, the emulsion composition comprises the copolymer surfactant at a concentration of about 1-30% (weight/volume), preferably about 20% weight/volume. In some embodiments, the immunotherapeutic agent is a toll-like receptor agonist.

In some embodiments of the current disclosure, pharmaceutical compositions are provided. In some embodiments, the pharmaceutical compositions comprise lipiodol, a hydrophilic surfactant, and one or more bioaffecting agents produced by: i) mixing the hydrophilic surfactant and one or more bioaffecting agents; ii) emulsifying the mixture of step i) in lipiodol for a sufficient time to produce an emulsion composition. In some embodiments, the one or more bioaffecting agents comprises a chemotherapeutic drug. In some embodiments, the chemotherapeutic drug is doxorubicin. In some embodiments, the one or more bioaffecting agents comprises an immunotherapeutic or immune boosting agent. In some embodiments, the immunotherapeutic is a toll-like receptor (TLR) agonist. In some embodiments, the emulsion composition comprises at least two bioaffecting agents, wherein the first bioaffecting agent is a cancer therapeutic and the second bioaffecting agent is an immune boosting or immunotherapeutic agent. In some embodiments, the viscosity of the composition is greater than about 80,000 mPaS, and wherein the composition has at least 500-fold increased stability as compared to a composition without the copolymer surfactant.

In some embodiments of the current disclosure, pharmaceutical compositions for use in locoregional delivery are provided. In some embodiments, the pharmaceutical compositions for use in locoregional delivery comprise: lipiodol emulsion, a copolymer surfactant, and one or more bioaffecting agents, wherein the composition has an increased stability, viscosity, locoregional deliver or combination thereof as compared to a composition without the copolymer surfactant. In some embodiments, the copolymer surfactant comprises a copolymer surfactant selected from the group consisting of: poloxamer 101, poloxamer 105, poloxamer, 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, Poloxamer 105 Benzoate, Poloxamer 182 Dibenzoate and Tween 80, poly(lactic-co-glycolic acid) (PLGA), and poly carprolacton (PCL) based amphiphilic block co-polymers. In some embodiments, the hydrophilic surfactant is poloxamer 188. In some embodiments, the copolymer surfactant has a concentration of about 1-30% (weight/volume), preferably about 20% weight/volume. In some embodiments, the one or more bioaffecting agents comprises a chemotherapeutic drug. In some embodiments, the chemotherapeutic drug is doxorubicin. In some embodiments, the one or more bioaffecting agents comprises an immunotherapeutic or immune boosting agent. In some embodiments, the immunotherapeutic is a toll-like receptor (TLR) agonist. In some embodiments, the one or more bioaffecting agents comprise doxorubicin and a toll-like receptor (TLR) agonist. In some embodiments, the viscosity of the composition is greater than about 80,000 mPaS. In some embodiments, the composition has at least 500-fold increased stability as compared to a composition without the copolymer surfactant.

### BRIEF DESCRIPTION OF THE DRAWINGS

FIGS. 1A-IF illustrate that PF 127 increases stability, and viscosity of Dox-Lipiodol emulsion. FIG. 1A is a schematic of PF127-incorporated Lipiodol emulsion preparation. FIG. 1B shows photographs of Lipiodol emulsions prepared with (20%) or without (0%) PF127 at various aqueous-to-lipid phase ratios. FIG. 1C shows microscopic images of Lipiodol emulsions 5 minutes after preparation at room temperature. FIG. 1D shows photographs of Lipiodol emulsions (1:2 phase ratio) in a capillary tube (75 mm in diameter) incubated at 37° C. for 24 hours. FIG. 1E shows Dox concentrations in the supernatant measured at various time points after Lipiodol emulsions were incubated at 37° C. for 5 minutes before the addition of PBS (with 10% FBS) pre-warmed to 37° C. Dox release profiles are shown with photographs of Lipiodol emulsions at 0 hour after PBS addition (n=3). Data are means #SEM. FIG. 1F shows viscosity of the emulsions at room temperature and 37° C.

FIGS. 2A-2E illustrate that PF 127 can be incorporated with Dox solution and does not affect its cytotoxicity. FIG. 2A. shows Dox and PF127 mixtures at various PF127 concentrations. Dox solution containing 20% PF127 was gelated at 37° C. FIG. 2B. shows stock solutions of Dox (45 mg/ml) and PF127 (30% w/v) mixed at a 1:2 volume ratio to prepare Dox solution containing 15 mg/ml Dox and 20% PF127. FIGS. 2C-E. show measurement of cytotoxicity of Dox with (20%) or without (0%) PF 127 measured using HepG2 cells. In FIG. 2C, HepG2 cells were incubated with Dox (10 μM) for 30 and 60 minutes and Dox uptake was analyzed using flow cytometry. FIG. 2D shows confocal microscopy images showing Dox uptake at 30 minutes after Dox incubation. In FIG. 2E, cell viability was measured 48 hours after incubation with Dox (0.01, 0.1, 1, 5, and 10 μM) using cell counting kit-8 (CCK-8) (n=5). Data are means±SEM.

FIGS. 3A-3B illustrate that PF127-incorporated lipiodol emulsions exhibit sustained drug release. In FIG. 3A, lipiodol emulsions prepared using aqueous solutions containing dextran or IgG were incubated at 37° C. for 5 minutes before the addition of PBS (with 10% FBS) pre-warmed to 37° C. (n=3). In FIG. 3B, LC beads in the range of 300-500 μm were incubated with Dox (500 μg), FITC-dextran (100 μg), or FITC-IgG (10 μg) dissolved in PBS at 4° C. for 4 hours. The supernatant was used to measure unloaded molecules and calculate loading efficacy (n =3). LC beads could not load other molecules but Dox. Data are means±SEM.

FIGS. 4A-4B illustrate catheter embolization of rabbit arteries. The catheter was introduced through the right femoral artery. The injection volume of the emulsion was approximately 0.5 ml. For imaging of vessels, Omnipaque diluted in saline was injected before and after embolization of arteries of the liver (FIG. 4A) or tumor (FIG. 4B). For the tumor model, a subcutaneous VX2 tumor model was used.

FIGS. 5A-5B illustrate that PF127 does not affect lipiodol CT. FIG. 5A shows ex vivo CT images of lipiodol emulsion with or without PF127. FIG. 5B shows CT conducted after TACE.

FIGS. 6A-6F illustrate that PF127 incorporation improves the safety and efficacy of conventional TACE. A week after tumor implantation, the rats were given TACE using Lipiodol emulsion with (20%) or without (0%) PF127. No treatment (NT) group did not receive any treatment. FIG. 6A shows plasma Dox concentration post-TACE (n=5 rats per group). FIG. 6B shows total systemic exposure of Dox as measured by area under curve from A. *** P<0.001, unpaired Student's t test. FIG. 6C shows body weight change post-TACE. * P<0.05, ** P<0.01, *** P<0.001 (versus NT), #P<0.05, ##P<0.01 (versus Dox+PF127), two-way repeated-measures ANOVA and Tukey's multiple comparisons test (n=5 rats per group). FIG. 6D shows representative MR images before and after TACE. The dotted line indicates tumor (n=6-7 rats per group). The scale bar indicates 1 cm. FIG. 6E shows relative tumor volume growth. * P<0.05, ** P<0.01, *** P<0.001 (versus NT), two-way repeated-measures ANOVA and Tukey's multiple comparisons test. FIG. 6F shows representative TUNEL images. The tissue samples were collected at 2 weeks post-TACE. The scale bar indicates 5 mm. Data are means±SEM. Data are pooled from two (FIGS. A-C) or three (FIGS. D-E) independent experiments.

FIG. 7 shows MR images of tumors in the NT group of FIG. 6. MR images were taken at 7 days after tumor implantation (pre-TACE). Additional MR images were taken after 1 and 2 weeks. The dotted line indicates tumor area. The scale bar indicates 1 cm. Data are pooled from three independent experiments.

FIG. 8 shows MR images of tumors in the Dox-Lipiodol group of FIG. 6. MR images were taken at 7 days after tumor implantation (pre-TACE). TACE was performed within the next two days and MR images were taken at 1 and 2 weeks after TACE. The dotted line indicates tumor area. The scale bar indicates 1 cm. Data are pooled from more than three independent experiments.

FIG. 9 shows MR images of tumors in the Dox-PF127-LPD group of FIG. 6. MR images were taken at 7 days after tumor implantation (pre-TACE). TACE was performed within the next two days and MR images were taken at 1 and 2 weeks after TACE. The dotted line indicates tumor area. The scale bar indicates 1 cm. Data are pooled from more than three independent experiments.

FIGS. 10A-10B illustrate that PF127 incorporation induces more cell death in HCC. Representative TUNEL-stained sections; regions indicated by the black box were shown in higher magnification. The scale bars indicate 5 mm (top) and 50 μm (bottom). Data are means±SEM.

FIGS. 11A-11C illustrate that transarterial delivery of CpG activates both innate and adaptive immunity in tumor. A week after tumor implantation, rats were given TACE with or without CpG. The rats were sacrificed 4 days after TACE for the evaluation of early immune responses (n=5 rats per group). FIG. 11A shows the excised tumors were digested to isolate single cells and then the cells were stained and assessed by flow cytometry.

Mean fluorescence intensity (MFI) of IFN-γ in dendritic cells (CD11b/c⁺), NK cells (CD161a⁺), and T cells (CD3⁺ CD4⁺ and CD3⁺CD8⁺). FIG. 11B shows the proportion of tumor-infiltrated CD8⁻ T cells. FIG. 11C is representative histology showing tumor-infiltrated CD8+ cells in tumor. The dotted line indicates tumor margin. The scale bar indicates 200 μm. * P<0.05, ** P<0.01, *** P <0.001, one-way ANOVA and Tukey's multiple comparisons test. Data are means±SEM. Data are pooled from two independent experiments.

FIG. 12 shows gating strategies for rat immune cells. Lymphocytes were gated by forward and side scatter, and singlets were gated. T lymphocytes were selected based on CD3 expression. The CD3+ population was further divided into CD4+ and CD8+ T cells based on their expression. NK cells were selected based on CD161α expression but no CD3 expression. The majority of CD161α+ cells were CD3 negative. Dendritic cells were selected based on CD11b/c expression.

FIG. 13 shows representative flow cytometric dot plots showing IFN-γ+ populations. The plots illustrate that CpG activates both innate and adaptive immunity in tumor.

FIG. 14 shows mean fluorescence intensity (MFI) of co-stimulatory molecules (CD86+) in dendritic cells. ** P<0.01, one-way ANOVA and Tukey's multiple comparisons test. Data are means±SEM. Data are pooled from two independent experiments.

FIGS. 15A-15E illustrate that transarterial delivery of CpG induces tumor-specific immunity. FIG. 15A shows representative MR images before and after TACE. The scale bar indicates 1 cm. FIG. 15B shows relative tumor volume growth. ** P<0.01, *** P<0.001 (versus NT), ##P<0.01 (versus TACE), two-way repeated-measures ANOVA and Tukey's multiple comparisons test (n=4-7). FIG. 15C is a schematic of splenocytes restimulation with tumor antigens. Splenocytes from treated and untreated groups were isolated and incubated with heat-killed tumor cells as a source of antigens. They were cultured together for 6 hours in the presence of Brefeldin A and IFN-γ secretion in CD8⁺ T cells were examined using flow cytometry. FIG. 15D shows representative mean fluorescence intensity (MFI) of IFN-γ in unstimulated and stimulated CD8⁺ T cells. FIG. 15E shows MFI of IFN-γ relative to no stimulation. * P<0.05, ** P <0.01, one-way ANOVA and Tukey's multiple comparisons test (n=4). Data are means±SEM. Data are pooled from two (FIG. 15B; TACE+CpG group, FIG. 15D, FIG. 15E) or three (FIG. 15B; NT and TACE groups) independent experiments.

FIG. 16 shows MR images of tumors in the TACIE group. MR images were taken at 7 days after tumor implantation (pre-TACE). TACIE was performed within the next two days and MR images were taken at 1 and 2 weeks after TACIE. The dotted line indicates tumor area. The scale bar indicates 1 cm. Data are pooled from two independent experiments.

FIGS. 17A-17C illustrate that transarterial CpG delivery does not induce systemic and local adverse events. FIG. 17A shows body weight change post-TACE. * P<0.05 (versus NT), two-way repeated-measures ANOVA and Tukey's multiple comparisons test (n=4-6 rats per group). FIGS. 17B-17C show plasma biochemistry analyses at day 4 and 14 post-TACE. FIG. 17B shows systemic inflammation assessed by measuring plasma levels of TNF-α and IFN-γ. FIG. 17C shows liver damage and function assessed by measuring plasma levels of AST, ALT, ALP, creatinine kinase, and total bilirubin. Data are means±SEM. Data are pooled from two independent experiments.

FIGS. 18A-18E illustrate that transarterial CpG delivery is effective in a DEN-induced HCC model. 10 weeks after DEN treatment, the rats were given either TACE or TACIE. No treatment (NT) group did not receive any treatment (N=4 per group). FIG. 18A shows representative macroscopic appearance and microscopic H&E-stained histology of the livers. The scale bar indicates 150 pm. FIGS. 18B-18C show tumor numbers (FIG. 18B) and largest tumor size (FIG. 18C) of HCC in each group. The tumors of 1 mm or larger were counted. * P<0.05, ** P <0.01, one-way ANOVA and Tukey's multiple comparisons test. FIG. 18D shows representative Sirius Red-stained histology of the livers. The scale bar indicates 150 pm. FIG. 18E shows fibrotic area measured from D. * P<0.05, ** P<0.01, one-way ANOVA and Tukey's multiple comparisons test. Data are means±SEM. The experiment was performed once.

### DETAILED DESCRIPTION OF THE INVENTION

In one aspect, the present invention provides methods of locoregional delivery and use of emulsion formulations that result in increased stabilization, viscosity and locoregional delivery of the composition comprising the bioaffecting agent, which results in surprisingly increased local regional delivery and reduced systemic side effects as compared to previous formulations.

In another aspect, the present invention provides formulations and methods incorporating a second bioaffecting agent, preferably an immune boosting or immunotherapeutic agent into the emulsion formulations to improve anti-tumor response.

Compositions, methods of use, and methods of preparing compositions for use in locoregional compound delivery are disclosed herein. It is to be understood that the disclosed compositions, methods of use, and methods of preparing said compositions are intended for use in any procedure where locoregional delivery of a compound or compounds is desired or intended. Therefore, the disclosed compositions, methods of use and methods of preparing compositions for use in locoregional delivery are intended for use in, by way of example but not by way of limitation, transarterial chemoembolization (TACE), intratumoral administration, or other locoregional delivery routes or procedures.

TACE is currently the standard of care for patients with intermediate stage hepatocellular carcinoma (HCC), which encompasses the widest class of patients with the disease. Clinical trials investigating the combination of intravenous immune checkpoint inhibitor (ICI) treatment with TACE are underway. However, the low response rate of ICI monotherapy in advanced HCC poses a main therapeutic challenge. Low mutational burden and immunosuppressive tumor microenvironment are the main challenges for the current ICI cancer immunotherapy and potential combinational ICI immunotherapies in HCC. There is accumulating data that locoregional delivery of immunotherapies to tumor can induce systemic antitumor immunity while minimizing systemic toxicity. The safety and efficacy of intratumoral administration of various immunomodulatory agents are under investigation in clinical trials. Since TACE can induce immunogenic cell death, concurrent delivery of immuno-boosting agents has tremendous potential to augment anti-tumor immunity.

In one embodiment, a strategy to incorporate immuno-boosting agents into the locoregional delivery, including TACE procedure, is provided with goals to promote antitumor immunity and response to ICI therapies. To solve the unmet need, the inventors engineered lipiodol emulsion with FDA-approved polymeric surfactants for safe delivery of various anti-cancer drugs for combination therapy. These compositions were found to have improved properties, e.g., increased stabilization and viscosity, allowing for increased local drug retention and reduction in systemic exposure, further reducing unintended side effects. In some embodiments, bead-based compositions are used in place of lipiodol to locoregionally deliver bioaffecting agents. Furthermore, it is to be understood that any materials that can be used to enhance drug delivery by improving, for example, stabilization, sustained release, and embolism may be used in the compositions, methods, and methods of production of the current disclosure.

The present inventions use copolymer surfactants (e.g., Pluronic F-127) when preparing lipiodol emulsion to help stabilize the emulsions and increase the viscosity, which facilitates local drug retention and minimize its systemic exposure. As demonstrated in the Examples, the incorporation of the surfactants significantly increased the stability and viscosity (810-fold at room temperature, 3600-fold at body temperature) of lipiodol emulsion in vitro. Further, in a N1-S1 rat HCC model, transarterial embolization using the formulation described herein significantly decreased systemic exposure of doxorubicin while improving local retention of the drug, which led to enhanced safety and efficacy of the TACE procedure. Furthermore, the incorporation of an immune-boosting agent (e.g., Toll-like receptor agonist) into the formulation induced significant activation of both innate and adaptive immunity in tumor compared to conventional TACE (doxorubicin only) without inducing systemic immune-related adverse events.

Lipiodol is used in TACE since it exhibits transient and plastic embolic effects, and it facilitates localized delivery of anticancer drugs into HCC. However, substantial instability and heterogeneity of the lipiodol-based emulsions and potential risk for systemic adverse events due to systemic leakage pose major setbacks in using it for TACE. Here, FDA-approved copolymer surfactants were used to stabilize and increase the viscosity of the emulsion. Using the new formulation, the inventors were able to achieve significantly decreased systemic exposure of doxorubicin while improving local retention of the drug, which led to enhanced safety and efficacy of the TACE procedure. Furthermore, the incorporation of an immune-boosting agent into the formulation induced significant activation of both innate and adaptive immunity in tumor compared to conventional TACE (doxorubicin only) without inducing systemic immune-related adverse events. Thus, the present disclosure provides compositions and methods for improved TACE procedures and compositions and methods that facilitate combination therapy to improve treatment of cancers by TACE, including HCC.

In one aspect of the current disclosure, pharmaceutical compositions for use in locoregional delivery are provided. In some embodiments, the compositions are for use in treating cancer using transarterial chemoembolization (TACE). In other embodiments, it may be used for intra-tumoral delivery. In some embodiments, the pharmaceutical compositions comprise: a lipiodol emulsion, a copolymer surfactant, and one or more bioaffecting agents, wherein the composition has an increased stability, increased viscosity, increased locoregional delivery or a combination of them as compared to a composition without the copolymer surfactant. In some embodiments, the compositions comprise: a bead based emulsion, a copolymer surfactant, and one or more bioaffecting agents, wherein the composition has an increased stability, increased viscosity, increased locoregional delivery or a combination of them as compared to a composition without the copolymer surfactant. Further, the compositions demonstrate a longer and improved local retention and a decreased systemic exposure. The compositions described herein can have an increased anti-cancer effect, as demonstrated by a reduction in tumor volume or size, reduction in number of tumor cells, reduction in tumor cell proliferation or growth, reduction in tumor metastasis, among other signs associated with anti-tumor effect (e.g., halt in tumor progression, reduction in one or more symptom associated with tumor, etc.).

As used herein, “cancer” or “tumor” refers to diseases, e.g., cell proliferative diseases, wherein an organism's cells grow uncontrollably and may spread to other locations in the organism (e.g., metastasize). By way of example, any cancer currently or that may be treated at some point with TACE may be contemplated by the present invention. Suitable cancers include, but are not limited to, hepatoma, hepatocellular carcinoma (primary liver cancer), cholangiocarcinoma (primary cancer of the bile ducts in the liver), metastasis in the liver from other cancers, including, for example, colon cancer, breast cancer, carcinoid tumors, neuroendocrine tumors, islet cell tumors of pancreas, ocular melanoma, vascular primary tumors, among others. In some embodiments, cancer refers to hepatocellular carcinoma (HCC).

As used herein, “transarterial chemoembolization (TACE)” or “transarterial embolization” refers to an image-guided, non-surgical procedure that is used to treat malignant lesions in the liver. The procedure uses an X-ray guided catheter to deliver both chemotherapy medication and embolization materials into the blood vessels that lead to the liver and to the tumor.

As used herein, “lipiodol” also known as “ethiodized oil” refers to a radio-opaque contrast agent comprising a combination of iodine and ethyl esters of poppy seed oil. The iodine is intercalated into the constituent fatty acids to produce a mixture of iodostearic and stearic-acid derived esters, (see, e.g., Yin, X. et al. Chemical shift MR imaging methods for the quantification of transcatheter lipiodol delivery to the liver: preclinical feasibility studies in a rodent model. (2012) Radiology. 263 (3): 714-22, incorporated by reference herein in its entirety).

As used herein, “copolymer surfactant” refers to a surfactant containing two hydrophilic portions and a hydrophobic group. In some embodiments, exemplary copolymer surfactants for use in the compositions and methods of the current disclosure may include, for example, poloxamer 101, poloxamer 105, poloxamer, 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, Poloxamer 105 Benzoate, Poloxamer 182 Dibenzoate, Tween 80, poly(lactic-co-glycolic acid) (PLGA), and poly carprolacton (PCL) based amphiphilic block co-polymers. In some embodiments, an exemplary copolymer surfactant for use in the disclosed compositions and methods is poloxamer 188.

As used herein, “poloxamer 188” may also refer to the compound with trade name “Pluronic R F-127”.

As used herein, “bioaffecting agent” refers to a compound, chemical, biologic, drug, or pharmaceutical compound that alters a biological process. In some embodiments, the bioaffecting agents are chemotherapeutics or other anti-cancer agents. In some embodiments, exemplary bioaffecting agents for use in the compositions and methods of the current disclosure include “immunotherapeutics” or “immune-boosting agents”.

As used herein, “immunotherapeutics,” “immunotherapies” or “immune-boosting agents” refers to molecules, chemicals and compounds that elicit an immune response and include, for example, checkpoint inhibitors, cancer vaccines, adoptive cell transfer therapies (ACT), and small molecules, among others. As used herein, these immune boosting agents refers to agents, molecules, and compounds that induce an increase the intensity, effectiveness, or duration of an immune response. In some embodiments, exemplary immune boosting compounds include “toll-like receptor agonists”. As used herein, “toll-like receptor agonist” refers to compounds that induce activation of one or more toll-like receptors. In some embodiments, exemplary bioaffecting agents/compounds include indolamine 2,3-dioxygenase (IDO) inhibitors. Other exemplary bioaffecting compounds include anti-PD-1 monoclonal antibodies, anti-PD-L1 monoclonal antibodies, anti-CTLA-4 monoclonal antibodies, anti-VISTA monoclonal antibodies, or other compounds targeting “immune checkpoint” molecules.

As used herein, “immune checkpoints” refers to proteins or peptides that regulate the activity of an immune response. For example, some immune checkpoints interfere with the ability of the immune system to mount an effective response. By way of example but not by way of limitation, immune checkpoints include the PD-1: PD-L1/PD-L2 axis.

As used herein, “immune checkpoint therapy” (“ICT”) refers to an intervention that is targeted to interfere with the normal function of “immune checkpoints.” In some embodiments, ICT comprises a treatment that interferes with the function of PD-1 or its ligands PD-L1 and PD-L2. In some embodiments, the ICT comprises a monoclonal antibody targeted to PD-1. In some embodiments, the monoclonal ICT therapy is selected from the group consisting of pembrolizumab, nivolumab, cemiplimab, atezolizumab, dostarlimab, durvalimab, and avelumab.

Checkpoint inhibitors that comprise anti-PD1 antibodies or anti-PDL1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8900587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi, see, e.g., U.S. Pat. Nos. 9,938,345 and 9,987,500), spartalizumab (PDR001, Novartis), camrelizumab (AiRuiKa, Hengrui Medicine Co.), sintillimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others and the like. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entireties with respect to the antibodies, their structure and sequences). Fragments of PD-1 or PD-L1 antibodies include those fragments of the antibodies that retain their function in binding PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

In some embodiments, exemplary bioaffecting agents for use in the compositions and methods of the current disclosure include “chemotherapeutics”.

As used herein, “chemotherapeutics” refers to compounds used to treat cancer including, but not limited to, cytotoxic agents, targeted therapies, and hormonal therapies. Exemplary chemotherapeutics for use in the compositions and methods of the current disclosure include: actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilon, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine. In some embodiments, exemplary chemotherapeutics for use in the compositions and methods of the current disclosure are daunorubicin, doxorubicin, epirubicin, idarubicin, valrubicin, Sorafenib, regorafenib, and Lenvatinib.

As used herein, an “emulsion” refers to a mixture of two or more liquids, which are normally immiscible, in which one is present as droplets, of microscopic or ultramicroscopic size, distributed throughout the other as a result of liquid-liquid phase separation.

As used herein, “stability” refers to the condition of a particular mixture remaining in the state which was achieved by the mixing of the constituent parts of the mixture. For example, in the case of a mixture of two compounds, stability will be reduced by an increased rate of dispersal of the two compounds. Thus, the stability of the mixture is negatively correlated with the rate of dispersal of the mixture.

As used herein, “viscosity” refers to a fluid's resistance to deformation at a given rate.

In another aspect of the current disclosure, methods of locoregionally delivering a compound to a subject in need of treatment for cancer in an amount effective to treat the cancer are provided. In some embodiments, the methods comprise i) preparing an emulsion composition comprising lipiodol, a copolymer surfactant, and one or more bioaffecting agents; ii) administering the emulsion composition directly into the cancer thereby delivering to the region of the cancer an effective amount of the bioaffecting agents.

As used herein, “locoregionally” refers to the condition of being limited to a local region of a subject's body.

For purposes of the present invention, “treating” or “treatment” describes the management and care of a subject for the purpose of combating the disease, condition, or disorder. Treating includes the administration of a compositions described herein when it is determined that the subject would be provided a benefit by the administration of the treatment to prevent the onset of the symptoms or complications, alleviating the symptoms or complications, or eliminating the disease, condition, or disorder.

The term “treating” can be characterized by one or more of the following: (a) the reducing, slowing or inhibiting the growth of cancer, including reducing slowing or inhibiting the growth of cancer cells; (b) preventing the further growth of tumors; (c) reducing or preventing the metastasis of cancer within a patient, and (d) reducing or ameliorating at least one symptom of the cancer. In some embodiments, the optimum effective amounts can be readily determined by one of ordinary skill in the art using routine experimentation.

A “subject in need thereof” as utilized herein may refer to a subject in need of treatment for a disease or disorder characterized by a tumor that is treatable by locoregional delivery of a bioaffecting agent, for example, by transarterial administration of a bioaffecting agent. A subject in need thereof may include a subject suffering from hepatocellular carcinoma (HCC). A subject in need thereof may include a subject in need of treatment by transarterial embolization. A subject in need thereof may include a subject in need of transarterial chemoembolization (TACE). A subject in need thereof may include a subject having a cancer for which treatment of the cancer would benefit from immune stimulation.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives.

As used herein, the terms “administering” and “administration” refer to any method of providing a pharmaceutical preparation to a subject. Such methods are well known to those skilled in the art and include, but are not limited to, transarterial administration, intra-arterial administration. Administration can be continuous or intermittent. In various aspects, a preparation can be administered therapeutically; that is, administered to treat an existing disease or condition. In a preferred embodiment, the administration is transarterial.

In a third aspect of the current disclosure, methods of treating cancer using TACE are provided. In some embodiments, the methods comprise administering a lipiodol emulsion, a copolymer surfactant, and one or more bioaffecting agents.

In a further aspect of the current disclosure, methods of activating an innate and/or adaptive immune response to a tumor in a subject in need thereof are provided. In some embodiments, the methods comprise: (i) administering a composition comprising at least one immune boosting and/or immunotherapeutic agent; and, wherein the composition activates an innate and/or adaptive immune response to the tumor as compared with a conventional composition. A conventional composition refers to a composition comprising lipiodol and a bioaffecting agent without the addition of a copolymer surfactant. Innate immune response refers to an innate immune interaction with tumor cells, which can include but is not limited to, for example, recognition by innate cell populations (NK cells, NKT cells, and γδ T cells) and also by dendritic cells and macrophages, CD8+ T cell responses and other pathways that stimulate the innate immune system, e.g., therapeutic stimulation of pattern recognition receptors (PRRs), such as Toll-like receptors (TLRs); the DNA sensing cGAS/STING pathway; nucleotide-binding oligomerization domain-like receptors (NLRs), such as NLRP3; and the retinoic acid-inducible gene-I (RIG-I)-like receptors (RLRs). Nonspecific and immediate immune responses are classified as innate due to their fast-acting nonspecific response against foreign antigens. Adaptive immune response involves the development of immunological memory due to specific forms of immune responses targeting the antigens with naïve lymphocytes, such as the T and B cells, gaining the ability to differentiate and mature into either effector T cells (CD4+ or CD8+ T cells) or antibody-secreting B cells (plasma cells), and is well understood in the art. CD4″ T cells CD4″ T cells can differentiate into several subsets of effector T cells such as T helper 1 (Th1) cells, T helper 2 (Th2) cells, or Tregs, with each of these subsets of CD4″ T effector cells can produce and secrete certain cytokines that modulate immune response accordingly. Similar to NK cells in innate immunity, naïve CD8⁺ T cells rely on MHC class I for maturation into effector cytotoxic T cells. CD8 T cells via the specific T cell receptor bind to the antigen/MHC class I complexes on the antigen-presenting cells (i.e., target cells) resulting in release of perforin and granzymes from CD8⁺ T cells and death of the target cell. Methods of determining the activation of an innate or adaptive immune response are well known in the art.

In another aspect of the current disclosure, a pharmaceutical composition comprising lipiodol, a hydrophilic surfactant, and one or more bioaffecting agents produced by: i) mixing the hydrophilic surfactant and one or more bioaffecting compounds; ii) emulsifying the mixture of step i) in lipiodol for a sufficient time to produce an emulsion composition is provided that may be used for locoregional delivery.

As used herein, “mixing” refers to the act of combining two or more substances. In some embodiments, mixing is accomplished by drawing up two liquids into two separate syringes, attaching the two syringes to two separate positions on a three-way stopcock, and passing the liquids through the three-way stopcock between two syringes with, by way of example but not by way of limitation, 30 pushes and pulls.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately.” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following Examples are illustrative and should not be interpreted to limit the scope of the claimed subject matter.

### Example 1

This Example demonstrates that the formation of a transarterial formulation for use in TACE that includes the addition of a copolymer surfactant to the lipiodol and a bioaffecting agent, e.g., chemotherapeutic agent doxycycline, increases local delivery and reduces toxic side effects of the composition when used to treat cancer. Further, this example demonstrates formulations and methods of activating an immune response to a tumor by trans-arterial administration of a formulation comprising lipiodol, a copolymer surfactant and an immune-boosting or immunotherapeutic agent (as the bioaffecting agent) allows for increase in an immune response to the tumor.

### INTRODUCTION

Systemic administration of immune checkpoint inhibitors (ICIs) targeting PD-1, PD-L1, or CTLA-4 is the mainstay in current cancer immunotherapy. However, advancement of the ICI immunotherapy is obstructed by the low response rate and immune-related adverse events (irAEs) in some cancer patients (1). Whereas combinations of ICIs can lead to higher response rates, the efficacy is coupled with higher systemic irAEs (2), which poses a main challenge in current immunotherapy. It requires strategies to safely combine immunotherapies. There is accumulating data that locoregional delivery of immunotherapies to tumors can induce systemic antitumor immunity while minimizing systemic toxicity (3-8). The application of locoregional immunotherapy has tremendous potential to improve therapeutic outcome of current immunotherapy.

Transarterial chemoembolization (TACE) is one of the locoregional therapies used for the treatment of hepatocellular carcinoma (HCC) (9). TACE is the standard of care for patients with intermediate stage HCC, which encompasses the widest class of patients with the disease. Considering the limited efficacy of systemic ICI monotherapy and the potential of combination (systemic+local) therapy in various cancers including advanced HCC (10-12), further development of synergistic combinations is required to promote the therapeutic efficacy. Based on the fact that TACE-mediated cell death induces release of tumor antigens (13), incorporating immunomodulatory agents into existing TACE emulsions can potentially facilitate in situ vaccination, promoting systemic antitumor immunity and response to immunotherapies.

Lipiodol exhibits transient and plastic embolic effect and facilitates localized delivery of Doxorubicin (Dox) or other anticancer drugs to HCC during TACE. However, substantial instability and heterogeneity of the lipiodol-based emulsions including therapeutic agents induce potential risk for systemic adverse events due to inevitable systemic leakage of loaded therapeutics (14). Therefore, the development of lipiodol-based formulations that can enhance stability and minimize systemic leakage may not only improve the current standard of care using chemotherapy drugs but also allow an opportunity for safe incorporation of potent immunotherapy agents into the procedure.

Pluronic F127 (PF 127) is an FDA-approved copolymer surfactant that can form thermoreversible hydrogel. Here, incorporation of PF 127 in the lipiodol-based emulsion was performed to investigate whether it can improve physicochemical properties of the emulsion since PF 127 is known to stabilize various forms of emulsions (15, 16). Preparation of PF127-incorporated lipiodol emulsions and an investigation of the safety and efficacy of transarterial delivery of doxorubicin (Dox)-loaded PF127-lipiodol emulsion were performed. CpG oligodeoxynucleotide for Toll-like receptor 9 (TLR9) immune adjuvant, which can develop an effective innate and adaptive immune response with TACE mediated antigens (17), was added to an optimized Dox loaded PF127-lipiodol emulsion (Dox-PF127-LPD) for transarterial chemoimmunoembolization (TACIE). In vivo feasibility of TACIE procedure was demonstrated by DSA-guided arterial embolization in rabbits and therapeutic response was evaluated in NIS1-and diethylnitrosamine (DEN)-HCC rat models. It was also shown that tumor response and immune investigation after the concurrent local delivery of CpG and TACE during TACIE demonstrated leveraged TACE-induced immunogenic tumor microenvironment and augmented systemic anti-tumor immunity.

### Results

### PF127 Improves Physicochemical Properties of Dox-Lipiodol Emulsion

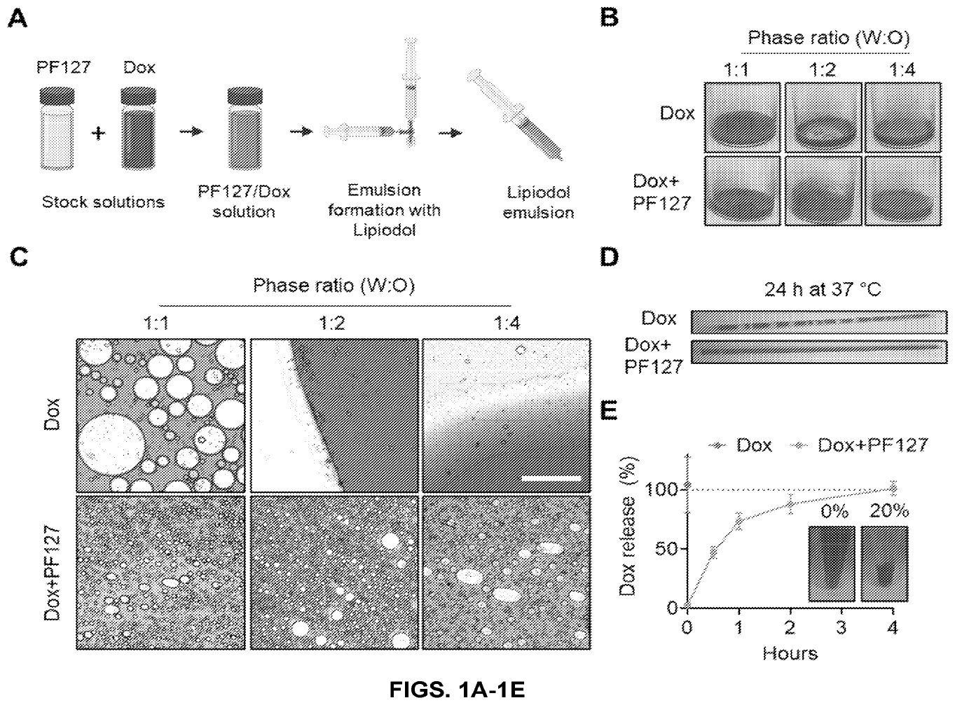

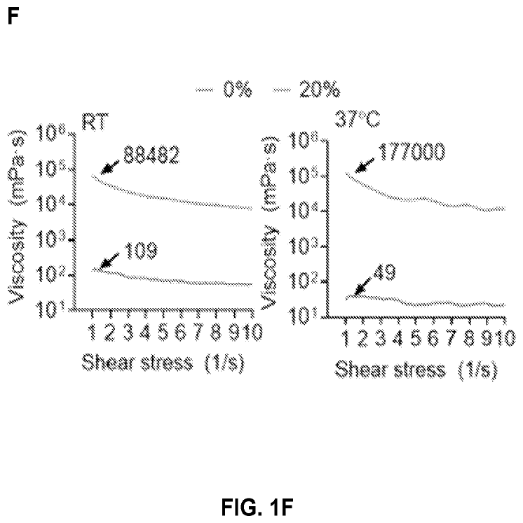

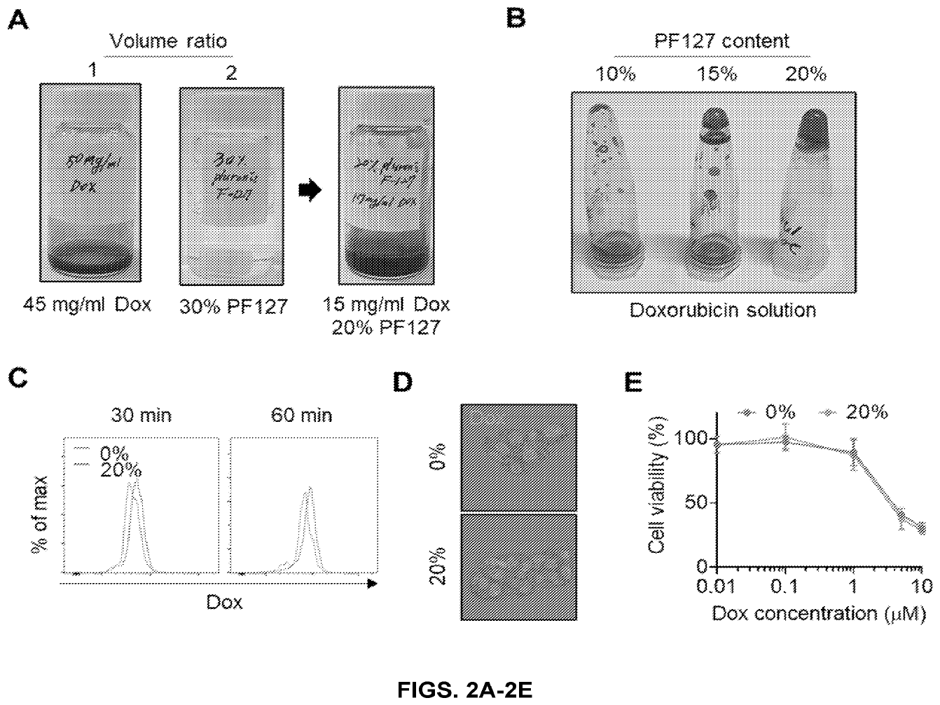

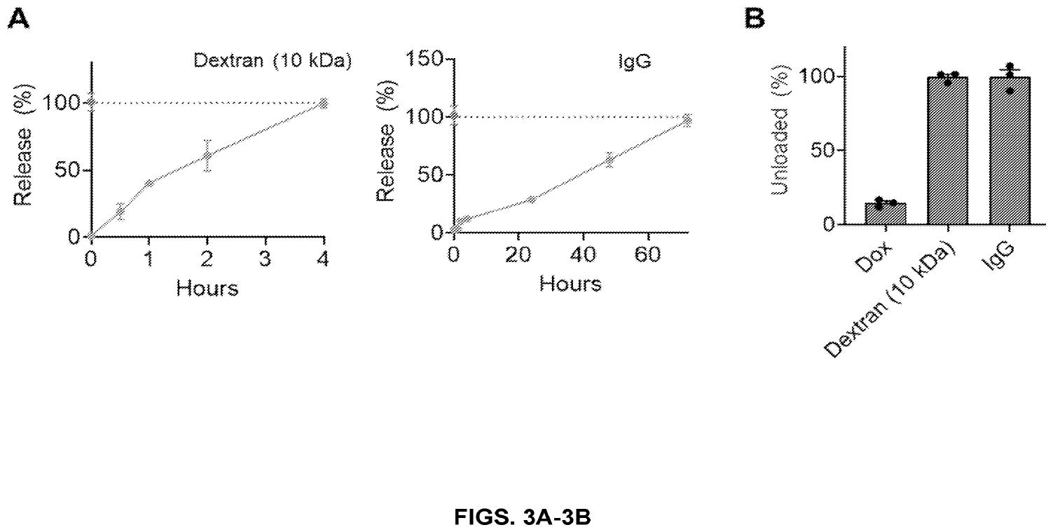

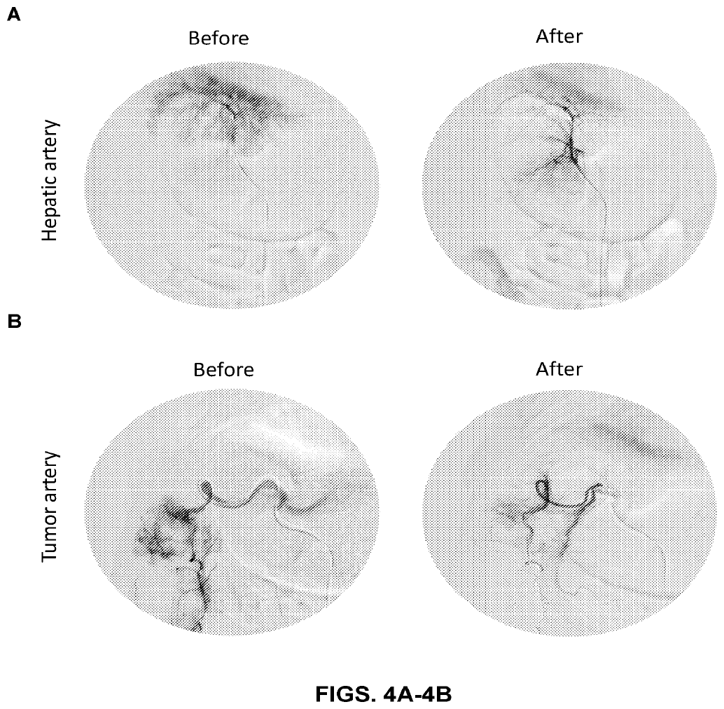

Dox-Lipiodol emulsions were prepared by a conventional protocol (18), where Dox (aqueous phase) and Lipiodol (oily phase) were mixed through a three-way stopcock between two syringes with 30 pushes and pulls (FIG. 1A). For the incorporation of PF 127, it was mixed in the aqueous phase with Dox to obtain a final concentration of 20% PF 127 (FIG. 2A), at which the PF127/Dox solution exhibited a gelation property at 37° C. (FIG. 2B). Cellular uptake and cytotoxicity of Dox were not affected by the addition of PF 127 (FIGS. 2C-E). Dox with or without PF 127 could be emulsified with Lipiodol in clinically relevant aqueous-to-lipid phase ratios of 1:1-1:4 (FIG. 1B). The Dox-lipiodol emulsion without PF 127 showed rapid phase separation within 5 minutes post-emulsification (FIG. 1C), as observed in previous reports (18, 19). However, Dox-lipiodol with PF127 (Dox-PF127-LPD) maintained stable emulsion with well-distributed aqueous droplets in the oil phase at room temperature (FIG. 1C). The enhanced stability of Dox-PF127-LPD was observed in an incubation at 37° C. for 24 hours in a capillary tube (FIG. 1D). The addition of PF 127 also allowed superior retention of various agents such as dextran and IgG including Dox at 37° C. (FIG. 1E and FIG. 3) (20). Then, in vivo feasibility of using Dox-PF127-LPD and its embolization effect could be demonstrated by transcatheter embolization of rabbit arteries (FIG. 4). The selected rabbit animal model has been commonly utilized for evaluating translational interventional procedures (21, 22). Hepatic artery catheterization and embolization using Dox-PF127-LPD were performed with DSA guidance (FIG. 4). Under DSA images from X-ray fluoroscopy, Dox-PF127-LPD could be successfully infused into the upstream at the PHA bifurcation into the right and left hepatic arteries (FIG. 4). The occluded arteries with infused Dox-PF127-LPD were visualized with Omnipaque contrasted DSA images before and after the infusion (FIG. 4). Overall, these data suggest that PF 127 can be successfully incorporated into Dox-Lipiodol emulsion. The demonstrated increased stability, drug retention and in vivo transcatheter intraarterial infusion of Dox-PF127-LPD suggest an effective form of lipiodol based agents for TACE and TACE based combination therapies of HCC.

### PF127 Improves the Safety and Efficacy of TACE

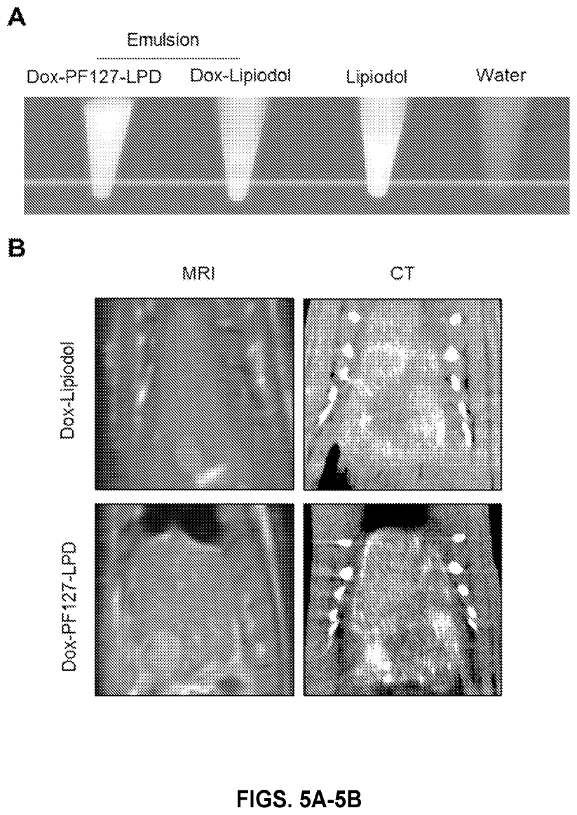

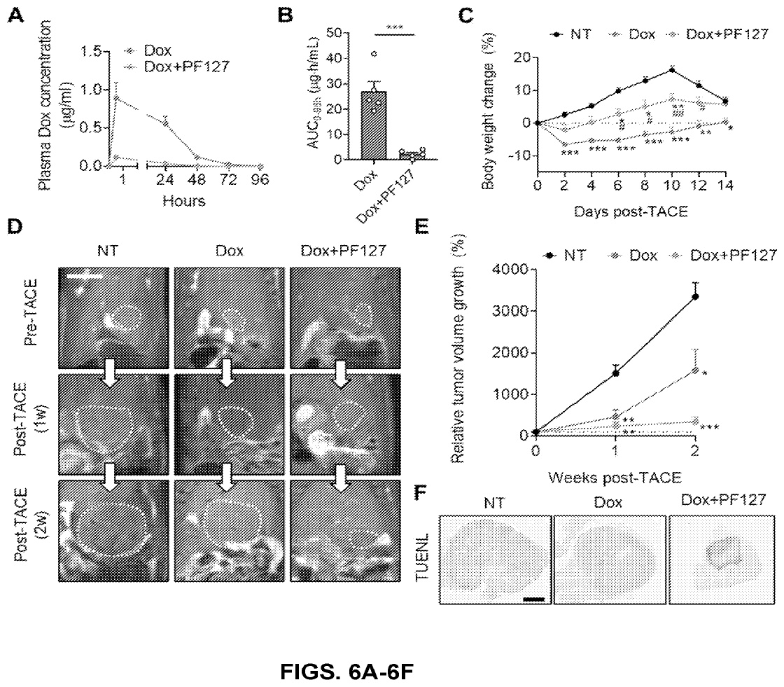

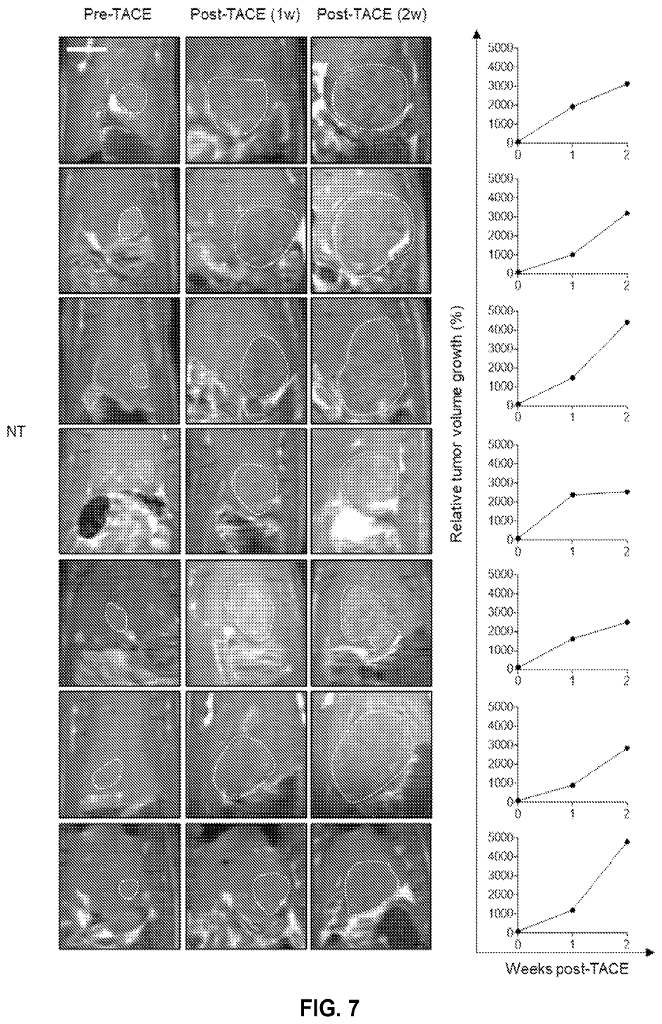

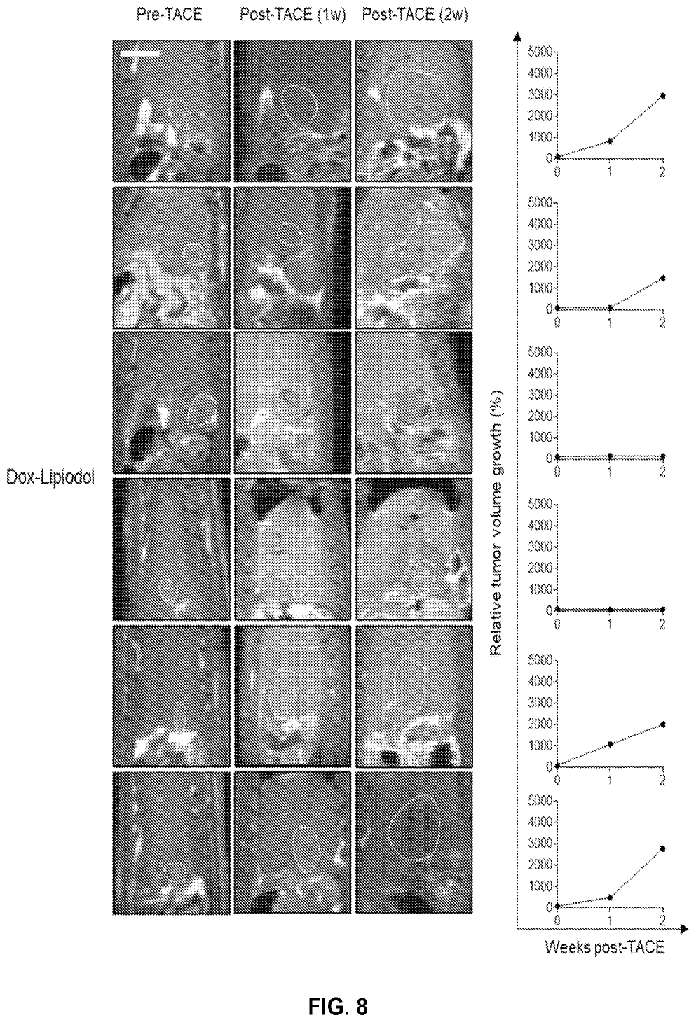

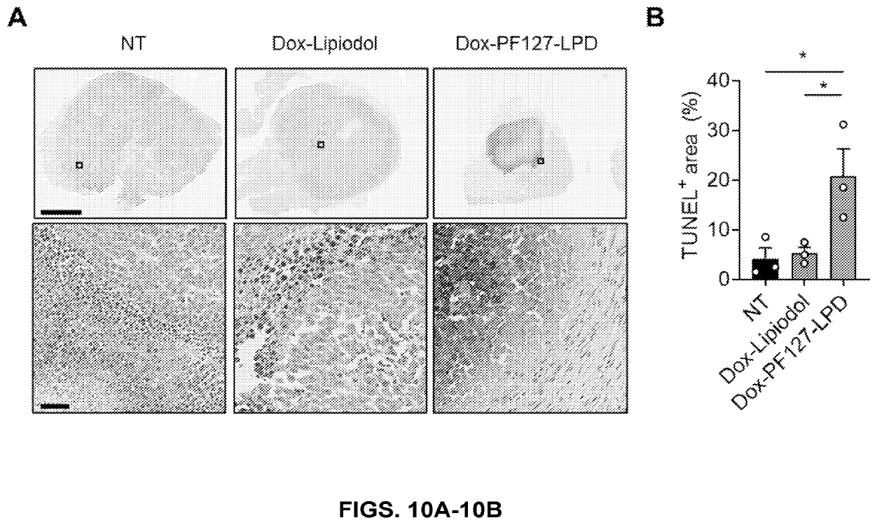

It was further investigated whether the improved physicochemical properties of Dox-PF 127-LPD emulsion in vitro can lead to enhanced safety and efficacy in vivo. For a tumor model, N1-S1 rat hepatoma was orthotopically implanted into the left hepatic lobe in Sprague Dawley (SD) rats (23). Baseline MRI obtained a week after implantation showed that the tumor volume reached-100 mm3 on average. TACE using Dox-lipiodol or Dox-PF127-LPD (Dox: 200 μg) was performed within 2 days after the baseline MRI scans. Successful transcatheter arterial delivery of samples was confirmed with enhanced CT contrast with radiopaque lipiodol, as shown by lipiodol accumulation (FIG. 5). Pharmacokinetics study showed that Dox-PF127-LPD significantly decreased Cmax and AUC of Dox compared to Dox-lipiodol (FIGS. 6A and B, and Table 1). Respective Cmax and AUC of Dox-PF127-LPD were 0.11±0.07 μg/ml and 2.34±1.52 μg h/ml, which were about 8˜12-folds lower than the results of Dox-lipiodol. Observation of body weight change revealed that both experimental groups exhibited post-treatment related body weight loss (FIG. 6C). However, the group treated with TACE using Dox-PF127-LPD exhibited less body weight loss and faster body weight recovery (FIG. 6C), due to the minimized systemic exposure of Dox (FIGS. 6A and B, and Table 1). Subsequent weekly MRI and MRI-based tumor volume measurement showed that Dox-PF 127-LPD led to superior tumor growth inhibition (FIGS. 2D and E and FIGS. 7-9). Both groups induced comparable tumor growth inhibition up to 1-week post-TACE. At 2-week post-TACE, the tumor growth of Dox-PF127-LPD TACE group was significantly suppressed while Dox-lipiodol TACE group showed exponential tumor growth during 2-week post-TACE. Histological analysis using TUNEL assay also revealed significantly enhanced cell death in the Dox-PF127-LPD group (FIG. 6F and FIG. 10). These data suggest that the altered physicochemical properties of the Dox-Lipiodol emulsion by the incorporation of PF127 could improve the therapeutic efficacy of TACE with reduced systemic toxicity of TACE.

TABLE 1
Pharmacokinetic parameters for each rat.

| | |
|---|---|
| Dox-Lipiodol | Dox-PF127-LPD |

| | Cₘₐₓ | AUC_{0-∞} | | Cₘₐₓ | AUC_{0-∞} |
|---|---|---|---|---|---|
| Rat # | (μg/ml) | (μg · h/ml) | Rat # | (μg/ml) | (μg · h/ml) |
| 1 | 1.24 | 27.57 | 1 | 0.18 | 4.071 |
| 2 | 0.52 | 19.38 | 2 | 0.09 | 2.222 |
| 3 | 0.40 | 22.6 | 3 | 0.01 | 0.138 |
| 4 | 1.49 | 41.96 | 4 | 0.08 | 3.425 |
| 5 | 0.78 | 23.63 | 5 | 0.18 | 1.824 |

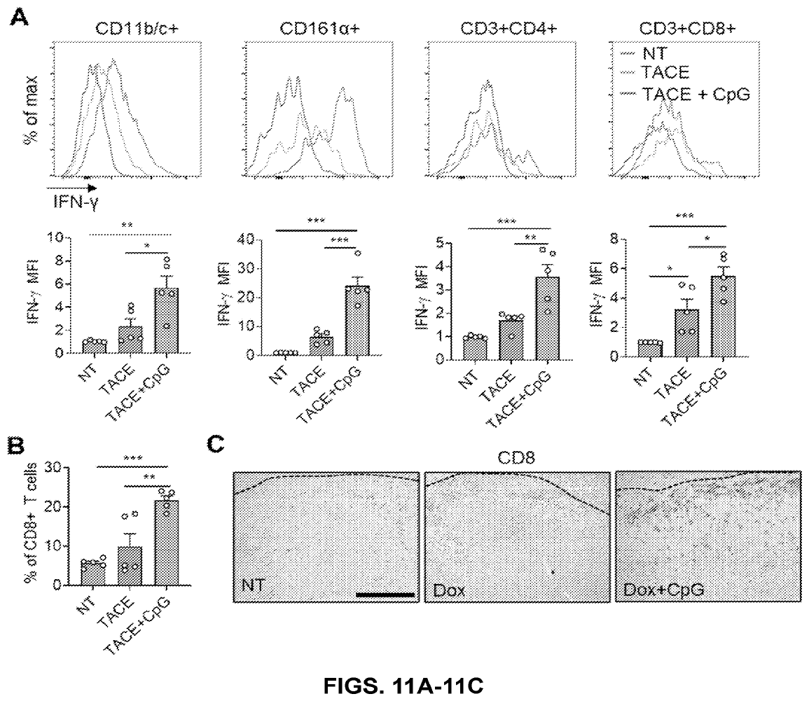

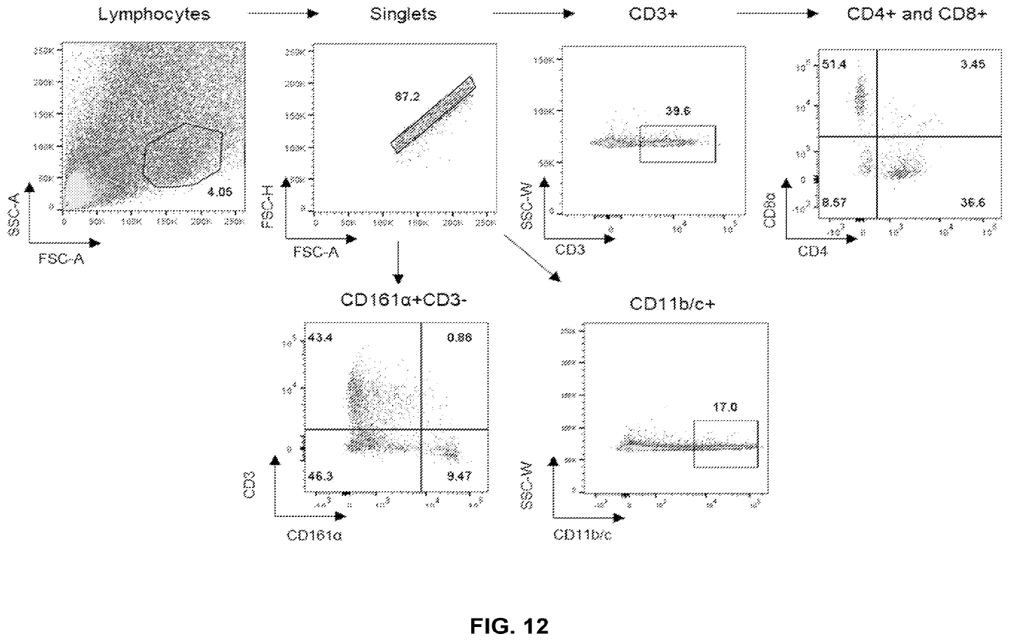

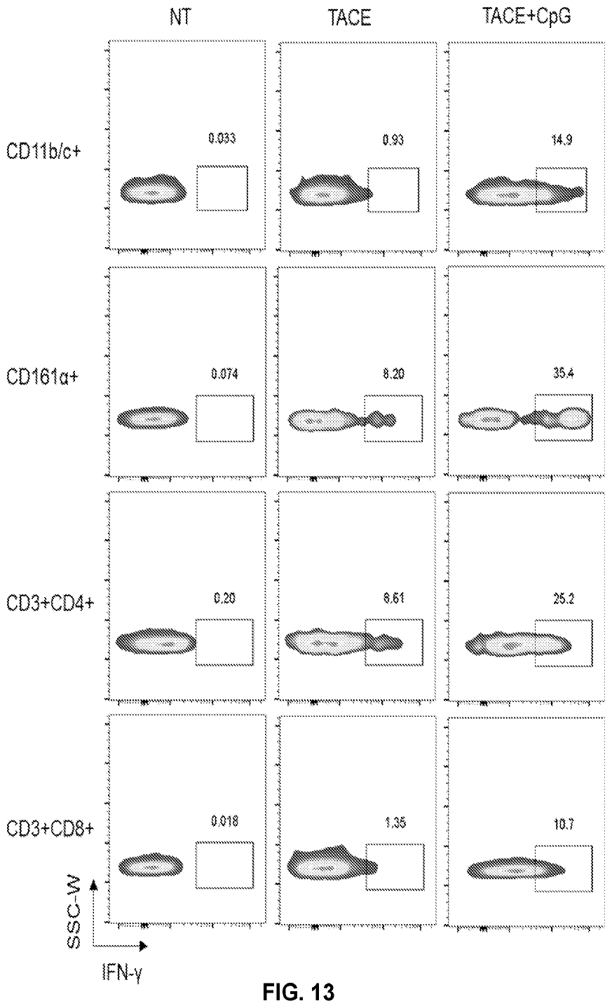

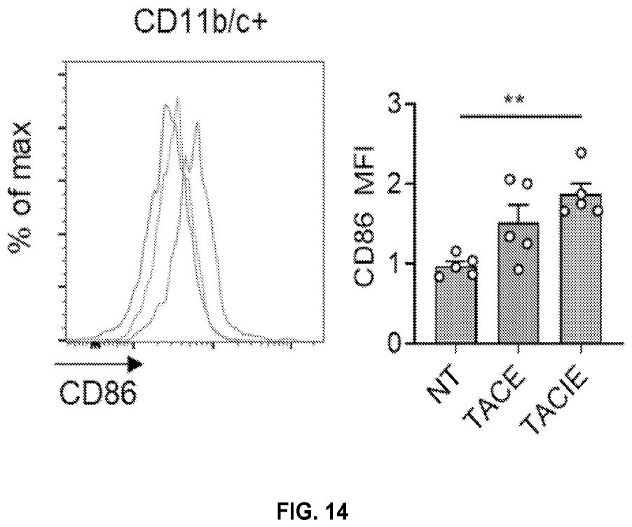

Since Dox-PF127-LPD facilitates safer delivery of drugs via transcatheter intra-arterial infusion, the inventors the Dox-PF127-LPD formulation could be exploited to co-deliver immunotherapeutic agents for TACIE. Toll-like receptors (TLRs) can signal the activation a variety of cells of the innate and adaptive immune system. Their local administration has been shown to effectively induce intratumoral immune activation and systemic anti-tumor T cell responses (7, 24). To investigate whether transcatheter intra-arterial co-delivery of TLRs with Dox induces similar immunological benefits, CpG oligonucleotide-a ligand for TLR9—was incorporated into the Dox-PF127-LPD by simply adding it in the aqueous phase with Dox. The intended doses of Dox and CpG in CpG-Dox-PF127-LPD were 200 μg and 20 μg, respectively. To assess early immune responses, the rats were treated with Dox-PF127-LPD (TACE group) or CpG-Dox-PF127-LPD (TACIE group) and the tumors were collected at 4 days after the treatments. The activation of immune system was manifested in the TACIE group by significant increase in interferon-γ (IFN-γ) secretion from antigen presenting cells (CD11b/c⁺), NK cells (CD161a⁺), and T cells (CD3⁺CD4⁺ and CD3⁺CD8⁺) (FIG. 11A, FIG. 12, and FIG. 13). Non-significant increase of the IFN-γ secretion in the TACE alone group implied its moderate activation of the immune system (25). Also, TACIE group demonstrated significantly increased tumor-infiltrated CD8+ T cells (FIG. 11B). The addition of CpG in TACE also significantly upregulated co-stimulatory molecule expression in antigen presenting cells (FIG. 14). Taken together, these data suggested that TACIE with CpG successfully induced robust local immune activation.

Transarterial CpG delivery activates innate and adaptive immunity in HCC

### Transarterial CpG Delivery Regresses Established HCC and Induces Systemic Tumor-Specific T Cell Immunity

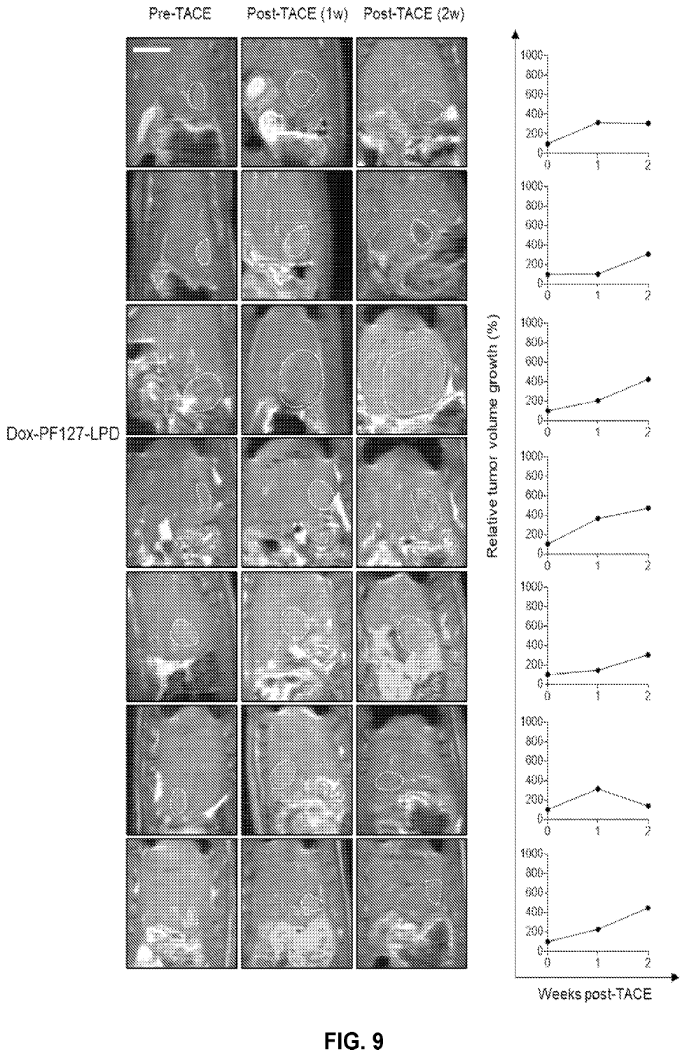

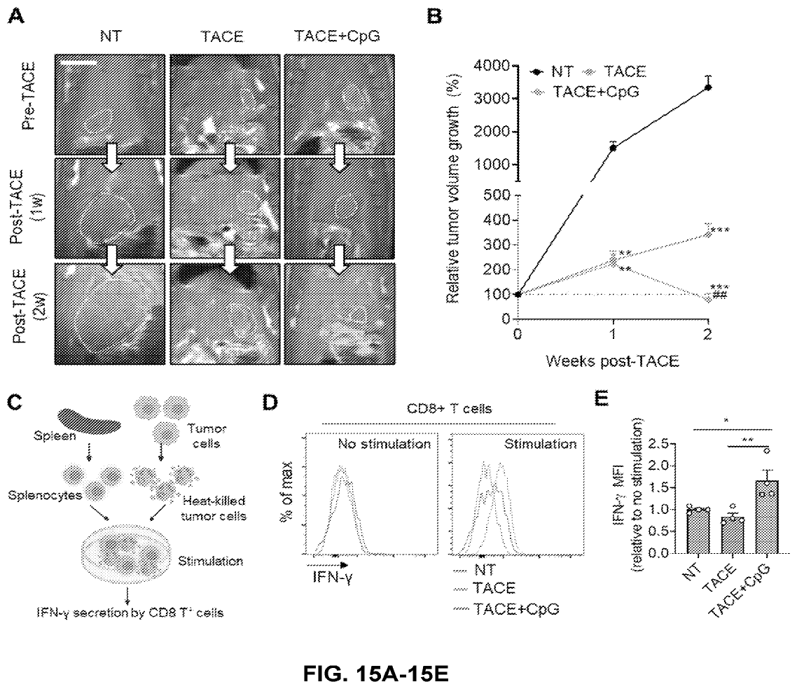

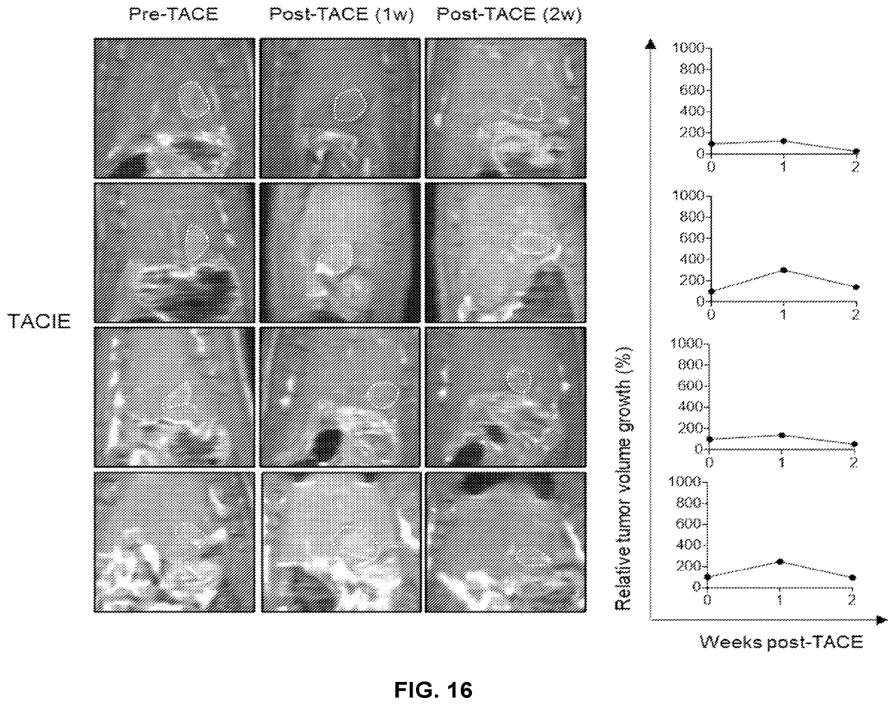

Since local immune activation was observed after TACIE combining TACE and intra-arterial delivered CpG, the therapeutic outcome and systemic anti-tumor immunity was evaluated. Radiographic assessment by MRI showed that the TACIE group exhibited comparable tumor size with TACE group at 1-week post-treatment (FIGS. 15A and 15B). However, at 2-week post-treatment, the TACIE group showed superior tumor growth inhibition compared to TACE group and regression compared to baseline (FIGS. 15A and 15B). The relative tumor volume compared to baseline at 2-week post-treatment was 341.8±115.4% and 79.4±49.5% in the TACE and TACIE groups, respectively. All the rats given TACE only (7/7) showed disease progression, while 75% of the rats in TACIE group given additional CpG (3/4) showed tumor regression (FIG. 9 and FIG. 16). To investigate the induction of systemic anti-tumor immunity, splenocytes were isolated from each treated and untreated groups and then incubated with heat-killed tumor cells as antigens (26, 27). It was then investigated whether there were tumor antigen-specific CD8⁺ T cells (FIG. 15C). Antigen stimulation resulted in an increase in its expression specifically in the TACIE group (FIGS. 15D and 15E), implying tumor-specific immunity. However, CD8⁻ T cells without antigen stimulation expressed a low level of proinflammatory cytokine IFN-γ in all groups. These data suggest that TACIE can effectively induce systemic anti-tumor immunity and significantly improve the therapeutic efficacy of HCC.

Transarterial CpG Delivery does not Induce Systemic or Local Toxicity

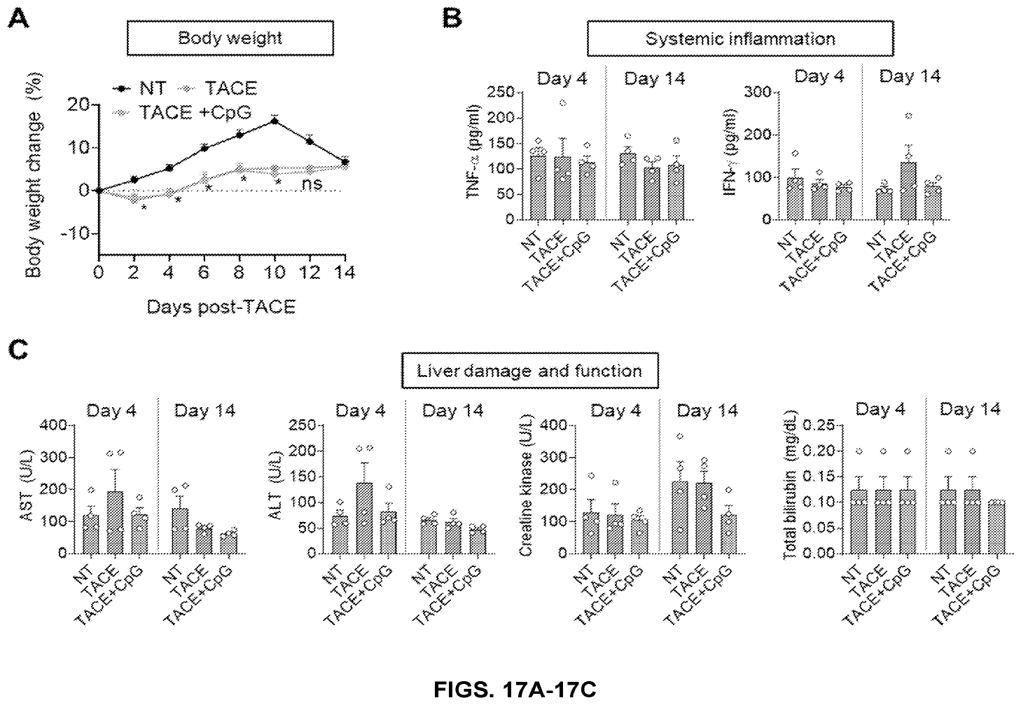

CpG is a robust immune adjuvant but systemic exposure of CpG is generally toxic and unacceptable in the clinic. Observation of body weight change revealed that additional CpG delivery of TACIE did not induce additional body weight loss and showed no significant difference in body weight change compared to TACE only group throughout 2-week post-treatment (FIG. 17A). Blood of treated and untreated rats were obtained at 3-days and 14-days post-treatment. Examination of serum proinflammatory cytokines TNF-α and IFN-γ revealed no significant difference across all group (FIG. 17B). Furthermore, serum concentrations of aspartate aminotransferase (AST), alanine aminotransferase (ALT), creatine kinase, and total bilirubin also showed no significant difference across all groups (FIG. 17C). These results show that TACIE does not cause systemic or local toxicity.

### Efficacy of Transarterial CpG is Confirmed in DEN-Induced HCC Model

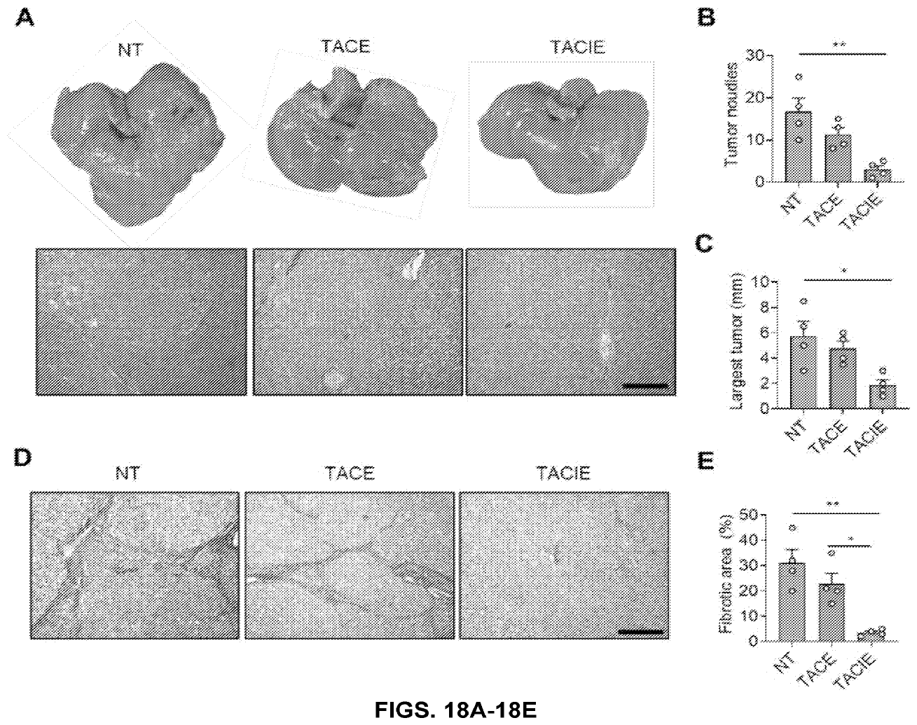

Finally, the broad utility and enhanced therapeutic efficacy of TACIE were validated in diethylnitrosamine (DEN)-induced rat HCC model. DEN causes liver tumors with a sequential progression of hepatitis, cirrhosis, and tumor formation, which closely mimics the development of human liver cancer. At 10 weeks after DEN treatment, laparotomy was performed to identify those with visible HCC nodules. Macroscopic observation revealed the development of tumor nodules especially in the non-treated group, which were confirmed through histological analysis (FIG. 18A). The number of HCC nodules larger than 1 mm in diameter was 17±6 in the no treatment group, 11±3 in the TACE group, and 3±2 in the TACIE group (FIG. 18B). The largest tumor size was 5.75±2.3 mm in the no treatment group, 4.75±1.2 mm in the TACE group, and 1.80±0.9 mm in the TACIE group (FIG. 18C). Also, fibrotic area measured by Sirius Red staining was 31.25±10.4% in the no treatment group, 22.75±8.6% in the TACE group, and 3.5 ±1.3% in the TACIE group (FIGS. 18D and 18E). These results show that TACIE local combination immunotherapy can effectively inhibit the development of HCC in DEN-induced model. Concurrent local CpG delivery with TACE as a form of TACIE can further enhance its therapeutic effect of TACE significantly.

### DISCUSSION

A practical strategy for augmenting immune activation after transarterial chemoembolization of HCC is shown herein. Incorporation of CpG into lipiodol based emulsion and its transarterial delivery performing TACIE induced robust local immune responses, which led to regression of HCC and formation of systemic anti-tumor immunity. Intratumoral CpG delivery, which can be considered as in situ vaccination, does not require information of the tumor antigens, and targets universal innate immune system. This approach using all clinical usable materials should be a convenient and powerful therapeutic approach that can be translated to the patients with HCC. Furthermore, the tumor-specific anti-cancer immunity by the local immunotherapy may improve the response to potential combination with other conventional ICI therapies.

Increased immune activation was observed in the TACE group compared to the no treatment group as assessed by IFN-γ secretion by various immune cells, CD8⁺ T cell recruitment, and expression of co-stimulatory receptors by APCs. Although the statistical significance of results was low, the tendency of enhancement of immune activation was consistent with previous studies (28). Dox agent used in TACE is known to induce immunogenic cell death (ICD) (29). Dox-TACE treated HCC patients have shown significant increase of ICD markers (30) and decreases of T-regs in the peripheral blood (31) with upregulated pro-inflammatory cytokine secretion (32). It is assumed that the superior therapeutic effect of TACIE (TACE+CpG-combination regimen) resulted from synergy by such immunomodulatory effects of TACE considering the limited therapeutic efficacy of CpG alone (7).

In many clinical and preclinical studies, in situ vaccination has been performed mainly via percutaneous intratumoral injection, requiring multiple injections during the treatment period (33). This poses drawbacks in terms of patient compliance, availability of a suitable injection site of the tumor, and requirements for additional imaging modalities. Herein, one-time local delivery of CpG was observed during TACIE, showing significant anti-cancer effect. This is reasonable since lipiodol emulsion allows retention and sustained release of drugs over weeks (34), which was further improved by the addition of PF127. Also, as TACE has been performed in the clinic, TACIE is not limited by injection-site and-parameters of tumor structures since it targets the artery that feeds the tumor. Lastly, TACIE is performed under well-established interventional oncology image-guidance. Therefore, TACIE incorporating CpG into TACE can be performed easily without significant changes in the current clinical procedures.

Risk and benefit considerations are of significant importance when combining immunotherapies with conventional therapies. TACE shows an objective response of about 50% based on RECIST and median survival with TACE ranges from 26 to 40 months, depending on stage of disease (35). Combining TACE with systemic anti-angiogenic agents (e.g., sorafenib) has not improved survival outcomes (36). The limited efficacy of ICI monotherapy in various cancers including advanced HCC (10, 11) suggests that combining ICIs with TACE would not be effective, and recently Nivolumab monotherapy was removed for the treatment of HCC in the clinic. Whereas combinations of ICIs can lead to higher response rates, the significantly higher incidence of irAEs from such regimens requires special attention to risk: benefit considerations in the patient group. However, locoregional immunotherapy performed in this study via intra-arterial injection did not induce local or systemic adverse events due to its limited distribution within tumor and low-dose requirement. The TACIE approach demonstrating robust local anti-cancer immunity and effective tumor response with safety suggests a new form of local immunotherapy for the treatment of HCC. The developed PF-127-lipiodol formulation also offers a great potential allowing various options for local immunotherapy combination with immune adjuvants, ICIs and chemotherapies.

Such safe integration of immunotherapy with conventional modality will be of critical importance in clinical trial design for various types of cancer.

### Materials and Methods

### Study Design

Two main objectives of this study were to develop a new lipiodol emulsion with better embolization properties and assess therapeutic efficacy of TACIE in rat models of HCC. Strategy for the first objective was to exploit PF 127, an FDA-approved amphiphilic block co-polymer, as stabilizers of lipiodol emulsion. It was found that PF 127 not only stabilizes the emulsion, as it prevented rapid phase separation, but also allows sustained release of incorporated drugs such as doxorubicin. The inventors confirmed the feasibility of using the formulation in rabbits as it showed good injectability and embolization effect. The improved safety and efficacy of the emulsion was demonstrated in a rat Nl-S1 HCC model. The strategy for the second objective was to incorporate CpG, a widely known and used TLR9 agonist to activate innate immune system, into the emulsion. It was hypothesized that TACE-induced antigen release can be leveraged by concurrent delivery of CpG and it can lead to effective local priming of immune cells. Two rat HCC models induced by implantation of Nl-S1 hepatoma cells and DEN were used to study the therapeutic efficacy of TACIE.

The general strategy for evaluation of immune response was to assess IFN-γ secretion using flow cytometry in immune populations as CpG directly impacts secretion of IFN-γ in dendritic cells and is a critical mediator of both innate and adaptive immunity. Formation of systemic anti-tumor immunity via splenocytes restimulation was assessed using heat-killed Nl-S1 tumor cells.

The sample sizes were selected on the basis of the results of pilot experiments so that relevant statistical tests could reveal significant differences between experimental groups. Animals that did not develop tumors or died because of surgery were excluded from the experiments. In therapy experiments using Nl-S1 HCC model, baseline tumor sizes were measured using MRI at 2 weeks post-implantation and then those with an established tumor were randomized into groups. The rats were given treatments within 2 days after baseline MRI. In therapy experiments using the DEN-induced model, laparotomy was performed after 10 weeks of DEN feeding to identify development of HCC. Those with established HCC were randomized into groups and given treatments. For each experiment, mice numbers, statistical tests, and numbers of experimental replicates are described in the figure legends. Investigators were not blinded during evaluation of the in vivo experiments.

### Reagents

CpG (ODN D-SL03) was purchased from Invivogen. Dox was purchased from LC Laboratories. PF127 was purchased from Sigma Aldrich. The following antibodies were used for flow cytometry: CD11b/c-BV421 (BD Biosciences; 743977), CD86-BV510 (BD Biosciences; 743212), PD-L1-FITC (Cell Signaling Technologies; 250485); CD3-BV605 (BD Biosciences; 563949), CD4-APC-Cy7 (BD Biosciences; 565432), CD8a-BV510 (BD Biosciences; 740139), CD161a-BV421 (BD Biosciences; 555008); IFN-Y-AF647 (BD Biosciences; 562213).

### Emulsion Preparation and Characterization

Water-in-oil emulsions were prepared with Lipiodol for the lipid phase and water for the aqueous phase with clinically relevant aqueous-to-lipid phase ratios of 1:1-1:4. Stock solutions of PF 127 (30% w/v), Dox (45 mg/ml), and Dox plus CpG (45 mg/ml Dox and 3 mg/ml CpG) were prepared in distilled water. To prepare PF127-containing lipiodol emulsion, the PF 127 stock solution was first mixed with a drug solution at a 2:1 volume ratio. For example, the PF127 and Dox plus CpG stock solutions were mixed to obtain a drug solution containing 20% w/v PF 127, 15 mg/ml Dox, and 1 mg/ml CpG. For the conventional formulation, distilled water without PF 127 was used. Then, Lipiodol and the drug solution were mixed using 30 back-and-forth pump exchanges through a 3-way stopcock. The formulations were loaded into a 1 ml syringe (Becton Dickinson) and injected through a 24-gauge, ¾-inch infusion catheter (Terumo) at a flow rate of 50 μl/s.

### Drug Release

Lipiodol-based emulsions at a 1:3 aqueous-to-lipid phase ratio were prepared using solutions of doxorubicin (45 mg/ml), FITC-IgG (400 μg/ml), and FITC-dextran (10 kDa) (1 mg/ml). 200 μl of each emulsion in a 1.5 ml microcentrifuge tube was incubated at 37° C. for 5 minutes before gently adding 1 ml of PBS containing 10% FBS pre-warmed to 37° C. The samples were maintained at 37° C. throughout. 20 μl of the supernatant was collected at varying time points to measure release of the molecules from the emulsion. To assess loading efficacy of LC beads, LC beads in the range of 300-500 μm (Biocompatibles, VE420GS) were used. 250 μl of the beads were drawn out of the vial and packing solution was removed. Then, the beads were incubated with doxorubicin (500 μg), FITC-IgG (10 μg), and FITC-dextran (100 μg) dissolved in PBS at 4° C. for 4 hours. The supernatant was used to measure unloaded molecules and calculate loading efficacy.

### Cell Line

The N1-S1 rat hepatoma cell line (ATCC, CRL-1604) was cultured in complete Dulbecco's modified Eagle's medium (DMEM) (Hyclone) supplemented with 10% fetal bovine serum (FBS) and 1% penicillin/streptomycin.

### Tumor Model and Intra-Arterial Therapy

All animal procedures were performed in accordance with protocols approved by the Institutional Animal Care and Use Committee. All rats were purchased from Envigo. For the NIS1 rat HCC tumor model, 300-350g male Sprague Dawley rats were implanted with 5×10⁶ N1S1 hepatoma cells in the left lateral lobe of the liver during mini-laparotomy procedures. At 7 days after tumor implantation when the tumor size reached approximately 5 mm in size, as measured by MR imaging, TACE was performed by infusing approximately 100 μl of Lipiodol emulsion into the left hepatic artery. Safety and efficacy of lipiodol emulsions with or without PF127 was evaluated. TACE was performed using lipiodol emulsion with (20%) or without (0%) PF 127 and the corresponding dose for doxorubicin was 200 μg. Pharmacokinetics of doxorubicin and body weight changes were measured for toxicity evaluation. MR imaging and immunohistochemistry were performed for efficacy evaluation. The effect of CpG incorporation into TACE was evaluated. TACE was performed using lipiodol emulsion with or without CpG and the corresponding doses for doxorubicin and CpG were 200 μg and 20 μg, respectively. Immune responses in the tumor were analyzed at 4 days and 14 days post-TACE using flow cytometry.

Immune-related adverse events were evaluated by measuring blood cytokine and blood tests at 4 and 14 days post-TACE. Tumor-specific immunity was analyzed using a splenocytes restimulation assay at 14 days post-TACE. For diethylnitrosamine (DEN)-induced HCC model, Wistar rats were given 0.01% DEN water bottles for a period of 10 weeks. Laparotomy was performed to identify liver tumor, and those with liver tumor were randomly grouped. Rats were sacrificed at 2 weeks post-TACE, and immediately after sacrifice, externally visible tumors larger than 1 mm were counted by stereo microscopy.

### MR Imaging

MR imaging studies were performed using a 7.0-T ClinScan high-field small animal MR imaging system with a commercial rat coil (Bruker Biospin, Billerica, Massachusetts).

Tumor volume was calculated as length×width²/2, where length represents the largest tumor diameter and width represents the perpendicular tumor diameter.

### Blood Collection

Blood was collected from the tail vein using a 23 G butterfly needle (Becton Dickinson). The tip of the needle was inserted into one of the lateral tail veins approximately 2 inches away from the tip of the tail. The rubber end of the butterfly needle was inserted into the vacuum blood collection tube coated with EDTA (Becton Dickinson; 367841). After collection, the needle was removed, and gentle pressure was applied to stop bleeding. The blood samples were centrifuged at 2,000×g for 5 min to separate plasma.

### FACS Analysis

Surface staining of cells was performed in phosphate-buffered saline (PBS) containing 1% FBS for 30 min at 4° C. For intracellular staining, the cells were resuspended in fixation/permeabilization (BD Bioscience) solution for 20 min at 4° C. before intracellular staining in permeabilization buffer for 30 min at 4° C. The stained cells were washed and resuspended in PBS for flow cytometric analysis. Flow cytometry was performed on a BD LSRFortessa (BD Biosciences).

### Splenocyte Restimulation

The spleen was collected in ice-cold complete RPMI medium, placed on a 70 pm cell strainer, and mashed using a syringe plunger through the strainer. Splenocytes were centrifuged at 350g for 10 min at 4° C. The cells were resuspended in ACK lysis buffer for the lysis of red blood cells. After resuspension in RPMI medium, cells were counted, diluted, and seeded onto a 24-well plate at a cell density of 5×10⁶ cells/ml. For splenocytes restimulation, p⁵ tumor cells heated at 45° C. for 1 h or irradiated under UVB lamp for 1 h were added. For IFN-γ detection, the cells were incubated with the tumor cells for 6 hours in the presence of 5 μg/ml Brefeldin A and flow cytometry was performed.

### Statistical Analysis

Prism 8.0 software was used for statistical analyses. Significant differences between two groups were analyzed using unpaired Student's t test. Analysis of variance (ANOVA) was used for greater than two groups with Tukey's test for multiple comparisons between all experimental groups. P values <0.05 were considered significant.

### REFERENCES

1. P. Darvin, S. M. Toor, V. Sasidharan Nair, E. Elkord, Immune checkpoint inhibitors: recent progress and potential biomarkers. Exp Mol Med 50, 1-11 (2018).
2. Larkin, V. Chiarion-Sileni, R. Gonzalez, J. J. Grob, C. L. Cowey, C. D. Lao, D. Schadendorf, R. Dummer, M. Smylie, P. Rutkowski, P. F. Ferrucci, A. Hill, J. Wagstaff, M. S. Carlino, J. B. Haanen, M. Maio, I. Marquez-Rodas, G. A. McArthur, P. A. Ascierto, G. V. Long, M. K. Callahan, M. A. Postow, K. Grossmann, M. Sznol, B. Dreno, L. Bastholt, A. Yang, L. M. Rollin, C. Horak, F. S. Hodi, J. D. Wolchok, Combined Nivolumab and Ipilimumab or Monotherapy in Untreated Melanoma. N Engl J Med 373, 23-34 (2015).
3. D. M. Francis, M. P. Manspeaker, A. Schudel, L. F. Sestito, M. J. O'Melia, H. T. Kissick, B. P. Pollack, E. K. Waller, S. N. Thomas, Blockade of immune checkpoints in lymph nodes through locoregional delivery augments cancer immunotherapy. Science Translational Medicine 12, (2020).
4. M. F. Fransen, T. C. van der Sluis, F. Ossendorp, R. Arens, C. J. Melief, Controlled local delivery of CTLA-4 blocking antibody induces CD8+ T-cell-dependent tumor eradication 06 and decreases risk of toxic side effects. Clin Cancer Res 19, 5381-5389 (2013).
5. F. Dammeijer, M. van Gulijk, E. E. Mulder, M. Lukkes, L. Klaase, T. van den Bosch, M. van Nimwegen, S. P. Lau, K. Latupeirissa, S. Schetters, Y. van Kooyk, L. Boon, A. Moyaart, Y. M. Mueller, P. D. Katsikis, A. M. Eggermont, H. Vroman, R. Stadhouders, R. W. Hendriks, J. V. Thusen, D. J. Grunhagen, C. Verhoef, T. van Hall, J. G. Aerts, The PD-1/PD-L1-Checkpoint Restrains T cell Immunity in Tumor-Draining Lymph Nodes. Cancer Cell, (2020).
6 M. F. Fransen, M. Schoonderwoerd, P. Knopf, M. G. Camps, L. J. Hawinkels, M. Kneilling, T. van Hall, F. Ossendorp, Tumor-draining lymph nodes are pivotal in PD-1/PD-L1 checkpoint therapy. JCI Insight 3, (2018).
7. I. Sagiv-Barfi, D. K. Czerwinski, S. Levy, I. S. Alam, A. T. Mayer, S. S. Gambhir, R. Levy, Eradication of spontaneous malignancy by local immunotherapy. Sci Transl Med 18, (2018).
8. J. D. Brody, W. Z. Ai, D. K. Czerwinski, J. A. Torchia, M. Levy, R. H. Advani, Y. H. Kim, R. T. Hoppe, S. J. Knox, L. K. Shin, I. Wapnir, R. J. Tibshirani, R. Levy, In situ vaccination with a TLR9 agonist induces systemic lymphoma regression: a phase I/II study. J Clin Oncol 28, 4324-4332 (2010).
9. R. Lencioni, T. de Baere, M. C. Soulen, W. S. Rilling, J. F. Geschwind, Lipiodol transarterial chemoembolization for hepatocellular carcinoma: A systematic review of efficacy and safety data. Hepatology 64, 106-116 (2016).
10. T. Yau, J. W. Park, R. S. Finn, A. L. Cheng, P. Mathurin, J. Edeline, M. Kudo, K. H. Han, J. J. Harding, P. Merle, O. Rosmorduc, L. Wyrwicz, E. Schott, S. P. Choo, R. K. Kelley, D. Begic, G. Chen, J. Neely, J. Anderson, B. Sangro, CheckMate 459: A randomized, multi-center phase III study of nivolumab (NIVO) vs sorafenib (SOR) as first-line (1L) treatment in patients (pts) with advanced hepatocellular carcinoma (aHCC). Ann Oncol 30, 31 874-+ (2019).
11. R. S. Finn, B. Y. Ryoo, P. Merle, M. Kudo, M. Bouattour, H. Y. Lim, V. Breder, J. Edeline, Y. Chao, S. Ogasawara, T. Yau, M. Garrido, S. L. Chan, J. Knox, B. Daniele, S. W. Ebbinghaus, E. Chen, A. B. Siegel, A. X. Zhu, A. L. Cheng, K.-. Investigators, Pembrolizumab As Second-Line Therapy in Patients With Advanced Hepatocellular Carcinoma in KEYNOTE-240: A Randomized, Double-Blind, Phase III Trial. Journal of Clinical Oncology 38, 193-+ (2020).
12. P. A. Ascierto, L. H. Butterfield, K. Campbell, B. Daniele, M. Dougan, L. A. Emens, S. Formenti, F. Janku, S. N. Khleif, T. Kirchhoff, A. Morabito, Y. Najjar, P. Nathan, K. Odunsi, A. Patnaik, C. M. Paulos, B. I. Reinfeld, H. D. Skinner, J. Timmerman, I. Puzanov, Perspectives in immunotherapy: meeting report from the “Immunotherapy Bridge” (Dec. 4th-5th, 2019, Naples, Italy). J Transl Med 19, 13 (2021).
13. J. P. Erinjeri, G. C. Fine, G. J. Adema, M. Ahmed, J. Chapiro, M. den Brok, R. Duran, S. J. Hunt, D. T. Johnson, J. Ricke, D. Y. Sze, B. B. Toskich, B. J. Wood, D. Woodrum, S. N. Goldberg, Immunotherapy and the Interventional Oncologist: Challenges and Opportunities-A Society of Interventional Oncology White Paper. Radiology 292, 25-34 (2019).
14. J. M. Idee, B. Guiu, Use of Lipiodol as a drug-delivery system for transcatheter arterial chemoembolization of hepatocellular carcinoma: A review. Crit Rev Oncol Hemat 88, 530-549 (2013).
15. M. A. Kabong, W. W. Focke, E. L. Du Toit, H. Rolfes, S. Ramjee, Breakdown mechanisms of oil-in-water emulsions stabilised with Pluronic F127 and co-surfactants. Colloid Surface A 585, (2020).
16. A. Torcello-Gomez, J. Maldonado-Valderrama, A. B. Jodar-Reyes, T. J. Foster, Interactions between Pluronics (F127 and F68) and Bile Salts (NaTDC) in the Aqueous Phase and the Interface of Oil-in-Water Emulsions. Langmuir 29, 2520-2529 (2013).
17. Q. Zhu, C. Egelston, A. Vivekanandhan, S. Uematsu, S. Akira, D. M. Klinman, I. M. Belyakov, J. A. Berzofsky, Toll-like receptor ligands synergize through distinct dendritic cell pathways to induce T cell responses: Implications for vaccines. Proceedings of the National Academy of Sciences 105, 16260-16265 (2008).
18. K. Kwak, B. Yu, S. K. Mouli, A. C. Larson, D. H. Kim, Sodium Cholate Bile Acid-Stabilized Ferumoxytol-Doxorubicin-Lipiodol Emulsion for Transcatheter Arterial Chemoembolization of Hepatocellular Carcinoma. J Vasc Interv Radiol 31, 1697-1705 64e1693 (2020).
19. M. Caine, T. Chung, H. Kilpatrick, Z. Bascal, S. Willis, Y. Tang, T. de Baere, M. Dreher, A. Lewis, Evaluation of novel formulations for transarterial chemoembolization:
   combining elements of Lipiodol emulsions with Drug-eluting Beads. Theranostics 9, 68 5626-5641 (2019).
20. B. Shriky, A. Kelly, M. Isreb, M. Babenko, N. Mahmoudi, S. Rogers, O. Shebanova, T. Snow, T. Gough, Pluronic F127 thermosensitive injectable smart hydrogels for controlled drug delivery system development. J Colloid Interface Sci 565, 119-130 (2020).
21. J. Chen, S. B. White, K. R. Harris, W. Li, J. W. Yap, D. H. Kim, R. J. Lewandowski, L. D. Shea, A. C. Larson, Poly(lactide-co-glycolide) microspheres for MRI-monitored delivery of sorafenib in a rabbit VX2 model. Biomaterials 61, 299-306 (2015).
22. R. C. Au-Khabbaz, Y.-H. Au-Huang, A. A. Au-Smith, K. D. Au-Garcia, R. P. Au-Lokken, R. C. Au-Gaba, Development and Angiographic Use of the Rabbit VX2 Model for Liver Cancer. JoVE, e58600 (2019).
23. Y. Guo, R. Klein, R. A. Omary, G. Y. Yang, A. C. Larson, Highly malignant intra-hepatic metastatic hepatocellular carcinoma in rats. Am J Transl Res 3, 114-120 (2010).
24. L. Hammerich, T. U. Marron, R. Upadhyay, J. Svensson-Arvelund, M. Dhainaut, S.Hussein, Y. Zhan, D. Ostrowski, M. Yellin, H. Marsh, A. M. Salazar, A. H. Rahman, B.D. Brown, M. Merad, J. D. Brody, Systemic clinical tumor regressions and potentiation of PD1 blockade with in situ vaccination. Nat Med 25, 814-824 (2019).
25. T. F. Greten, M. Mauda-Havakuk, B. Heinrich, F. Korangy, B. J. Wood, Combined locoregional-immunotherapy for liver cancer. J Hepatol 70, 999-1007 (2019).
26. D. Montagna, R. Maccario, F. Locatelli, V. Rosti, Y. Yang, P. Farness, A. Moretta, P. Comoli, E. Montini, A. Vitiello, Ex vivo priming for long-term maintenance of antileukemia human cytotoxic T cells suggests a general procedure for adoptive 89 immunotherapy. Blood 98, 3359-3366 (2001).
27. A. Uchida, M. Moore, E. Klein, Autologous Mixed Lymphocyte-Tumor Reaction and Autologous Mixed Lymphocyte-Reaction 2. Generation of Specific and Non-Specific Killer T-Cells Capable of Lysing Autologous Tumor. Int J Cancer 41, 651-656 (1988).
28. J. M. Llovet, T. De Baere, L. Kulik, P. K. Haber, T. F. Greten, T. Meyer, R. Lencioni, Locoregional therapies in the era of molecular and immune treatments for hepatocellular carcinoma. Nat Rev Gastroenterol Hepatol 18, 293-313 (2021).
29. G. Kroemer, L. Galluzzi, O. Kepp, L. Zitvogel, Immunogenic Cell Death in Cancer Therapy. Annu Rev Immunol 31, 51-72 (2013).
30. N. Kohles, D. Nagel, D. Jungst, P. Stieber, S. Holdenrieder, Predictive value ofimmunogenic cell death biomarkers HMGB1, sRAGE, and DNase in liver cancer patients receiving transarterial chemoembolization therapy. Tumour Biol 33, 2401-2409 (2012)
31. J. Liao, J. Xiao, Y. Zhou, Z. Liu, C. Wang, Effect of transcatheter arterial chemoembolization on cellular immune function and regulatory T cells in patients with hepatocellular carcinoma. Mol Med Rep 12, 6065-6071 (2015).
32. M. J. Kim, J. W. Jang, B. S. Oh, J. H. Kwon, K. W. Chung, H. S. Jung, D. W. Jekarl, S. Lee, Change in inflammatory cytokine profiles after transarterial chemotherapy in patients with hepatocellular carcinoma. Cytokine 64, 516-522 (2013).
33. W. X. Hong, S. Haebe, A. S. Lee, C. B. Westphalen, J. A. Norton, W. Jiang, R. Levy, Intratumoral Immunotherapy for Early-stage Solid Tumors. Clin Cancer Res 26, 3091-093099 (2020).
34. M. D. Burgio, R. Sartoris, C. Libotean, M. Zappa, A. Sibert, V. Vilgrain, M. Ronot, Lipiodol retention pattern after TACE for HCC is a predictor for local progression in lesions with complete response. Cancer Imaging 19, (2019).
35. A. Villanueva, Hepatocellular Carcinoma. N Engl J Med 380, 1450-1462 (2019). 36 T. Meyer, R. Fox, Y. T. Ma, P. J. Ross, M. W. James, R. Sturgess, C. Stubbs, D. D.Stocken, L. Wall, A. Watkinson, N. Hacking, T. R. J. Evans, P. Collins, R. A. Hubner, D. Cunningham, J. N. Primrose, P. J. Johnson, D. H. Palmer, Sorafenib in combination with transarterial chemoembolisation in patients with unresectable hepatocellular carcinoma (TACE 2): a randomised placebo-controlled, double-blind, phase 3 trial. Lancet Gastroenterol Hepatol 2, 565-575 (2017).

1. A method of locoregionally delivering one or more bioaffecting agents to a subject in need of treatment for a cancer, the method comprising:
i) preparing an emulsion composition comprising lipiodol, a copolymer surfactant, and the one or more bioaffecting agents;
ii) administering the emulsion composition locoregionally to the subject at a region of the cancer, thereby delivering an effective amount of the bioaffecting agents.

2. The method of claim 1, wherein the emulsion composition is delivered by transarterial administration.

3. The method of claim 1, wherein the emulsion composition is delivered by intratumoral administration.

4. The method of any of claims 1-3, wherein the one or more bioaffecting agents comprises a chemotherapeutic drug.

5. The method of claim 4, wherein the chemotherapeutic drug is doxorubicin.

6. The method of any of claims 1-5, wherein the one or more bioaffecting agents comprises an immunotherapeutic or immune boosting agent.

7. The method of any of claims 1-6, wherein the immunotherapeutic is a toll-like receptor (TLR) agonist.

8. The method of claim 1, wherein the emulsion composition comprises at least two bioaffecting agents, wherein the first bioaffecting agent is a cancer therapeutic and the second bioaffecting agent is an immune boosting or immunotherapeutic agent.

9. The method of claim 8, wherein the one or more bioaffecting agents comprise doxorubicin and a toll-like receptor (TLR) agonist.

10. The method of any one of claim 1-9, wherein the emulsion composition comprises the copolymer surfactant at a concentration of about 1-30% (weight/volume), preferably about 20% weight/volume.

11. The method of any one of claims 1-10, wherein the emulsion composition has at least 500-fold increased stability as compared to a composition without the copolymer surfactant.

12. The method of any one of claims 1-11, wherein the one or more bioaffecting agents remain localized to the tumor for a suitable amount of time to treat the cancer.

13. A method of treating a cancer, the method comprising:
(i) administering an emulsion composition comprising lipiodol, a copolymer surfactant, and one or more bioaffecting agents in an amount effective to treat the cancer.

14. The method of claim 13, wherein the emulsion composition is delivered by transarterial administration.

15. The method of claim 13, wherein the emulsion composition is delivered by intratumoral administration.

16. The method of any of claims 13-15, wherein the emulsion composition comprises the copolymer surfactant at a concentration of about 1-30% (weight/volume), preferably about 20% weight/volume.

17. The method of any of claims 13-16, wherein the one or more bioaffecting agents comprises a chemotherapeutic drug.

18. The method of claim 17, wherein the chemotherapeutic drug is doxorubicin.

19. The method of any of claims 13-18, wherein the one or more bioaffecting agents comprises an immunotherapeutic or immune boosting agent.

20. The method of claim 19, wherein the immunotherapeutic is a toll-like receptor (TLR) agonist.

21. The method of any one of claims 13-20, wherein the emulsion composition comprises at least two bioaffecting agents, wherein the first bioaffecting agent is a cancer therapeutic and the second bioaffecting agent is an immune boosting or immunotherapeutic agent.

22. The method of claim 21, wherein the one or more bioaffecting agents comprise doxorubicin and a toll-like receptor (TLR) agonist.

23. The method of any one of claims 13-22, wherein the emulsion composition is made by the method comprising:
i) mixing the hydrophilic surfactant and one or more bioaffecting agents;
ii) emulsifying the mixture of step i) in lipiodol for a sufficient time to produce an emulsion composition.

24. A method of activating an innate and/or adaptive immune response to a tumor in a subject in need thereof, the method comprising:
(i) administering an emulsion composition comprising lipiodol, a hydrophilic surfactant and one or more bioaffecting agents, wherein the one or more bioaffecting agents comprises at least one immune boosting or immunotherapeutic agents; and wherein the composition activates an innate and/or adaptive immune response to the tumor as compared with conventional composition.

25. The method of claim 24, wherein the emulsion composition is delivered by transarterial administration.

26. The method of claim 24, wherein the emulsion composition is delivered by intratumoral administration.

27. The method of any of claims 24-26, wherein the emulsion composition comprises the copolymer surfactant at a concentration of about 1-30% (weight/volume), preferably about 20% weight/volume.

28. The method of any of claims 24-27, wherein the immunotherapeutic agent is a toll-like receptor agonist.

29. A pharmaceutical composition comprising lipiodol, a hydrophilic surfactant, and one or more bioaffecting agents produced by:
i) mixing the hydrophilic surfactant and one or more bioaffecting agents;
ii) emulsifying the mixture of step i) in lipiodol for a sufficient time to produce an emulsion composition.

30. The composition of claim 29, wherein the one or more bioaffecting agents comprises a chemotherapeutic drug.

31. The composition of claim 30, wherein the chemotherapeutic drug is doxorubicin.

32. The composition of any of claims 29-31, wherein the one or more bioaffecting agents comprises an immunotherapeutic or immune boosting agent.

33. The composition of claim 32, wherein the immunotherapeutic is a toll-like receptor (TLR) agonist.

34. The composition of any one of claims 29-33, wherein the emulsion composition comprises at least two bioaffecting agents, wherein the first bioaffecting agent is a cancer therapeutic and the second bioaffecting agent is an immune boosting or immunotherapeutic agent.

35. The composition of any of claims 29-34, wherein the viscosity of the composition is greater than about 80,000 mPaS, and wherein the composition has at least 500-fold increased stability as compared to a composition without the copolymer surfactant.

36. A pharmaceutical composition for use in locoregional delivery comprising: lipiodol emulsion, a copolymer surfactant, and one or more bioaffecting agents, wherein the composition has an increased stability, viscosity, locoregional deliver or combination thereof as compared to a composition without the copolymer surfactant.

37. The composition of claim 36, wherein the copolymer surfactant comprises a copolymer surfactant selected from the group consisting of: poloxamer 101, poloxamer 105, poloxamer, 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, Poloxamer 105 Benzoate, Poloxamer 182 Dibenzoate and Tween 80, poly(lactic-co-glycolic acid) (PLGA), and poly carprolacton (PCL) based amphiphilic block co-polymers.

38. The composition of any of claims 36-37, wherein the hydrophilic surfactant is poloxamer 188.

39. The composition of any of claims 36-38, wherein the copolymer surfactant has a concentration of about 1-30% (weight/volume), preferably about 20% weight/volume.

40. The composition of any of claims 36-39, wherein the one or more bioaffecting agents comprises a chemotherapeutic drug.

41. The composition of any of claims 36-40, wherein the chemotherapeutic drug is doxorubicin.

42. The composition of claim 40, wherein the one or more bioaffecting agents comprises an immunotherapeutic or immune boosting agent.

43. The composition of any of claims 36-42, wherein the immunotherapeutic is a toll-like receptor (TLR) agonist.

44. The composition of any of claims 36-43, wherein the one or more bioaffecting agents comprise doxorubicin and a toll-like receptor (TLR) agonist.

45. The composition of any of claims 36-44, wherein the viscosity of the composition is greater than about 80,000 mPaS.

46. The composition of any one of claims 36-45, wherein the composition has at least 500-fold increased stability as compared to a composition without the copolymer surfactant.
